(19) United States
(12) Patent Application Publication
(10) Pub. No.: US 2015/0290354 A1
(43) Pub. Date: Oct. 15, 2015

(54) NONWOVEN FIBER MATERIALS

(71) Applicant: NORTH CAROLINA STATE UNIVERSITY, Raleigh, NC (US)

(72) Inventors: Elizabeth G. Loboa, Cary, NC (US); Behnam Pourdeyhimi, Cary, NC (US); Mahsa Mohiti Asli, Raleigh, NC (US)

(73) Assignee: NORTH CAROLINA STATE UNIVERSITY, Raleigh, NC (US)

(21) Appl. No.: 14/437,624

(22) PCT Filed: Oct. 22, 2013
(86) PCT No.: PCT/US2013/066030

§ 371 Date: Apr. 22, 2015

Related U.S. Application Data
(60) Provisional application No. 61/716,820, filed on Oct. 22, 2012.
(60) Provisional application No. 61/846,396, filed on Jul. 15, 2013.

(51) Int. Cl. A61L 15/42; A61L 15/26; A61L 15/44; A61L 15/40; D04H 1/4382; D04H 1/728

(52) U.S. Cl. CPC A61L 15/425; D04H 1/4382; D04H 1/728; A61L 15/44; A61L 15/40; A61L 15/26; D10B 2509/022; A61L 2300/61; A61L 2300/104; A61L 2300/30; A61L 2300/21; A61L 2300/41

(57) ABSTRACT

The present invention provides electrospun fibrous materials with various potential applications in the healthcare industry. Unique fiber morphologies are provided, which can allow the fibrous materials to exhibit a range of desirable properties. The electrospun fibrous materials are advantageously biocompatible and may be tailored for certain specific applications, e.g., by the incorporation of one or more therapeutic agents. Exemplary materials described herein can be employed in controlled, localized drug delivery, tissue engineering, and wound healing applications.

### FIELD OF THE INVENTION

The present invention relates to electrospun polymeric fiber structures for various biological applications and methods of production and use thereof.

### BACKGROUND OF THE INVENTION

The field of healthcare has long benefited from the use of nonwoven structures for wound healing applications due to their absorbent properties, facile processing schemes, and relative cost-effectiveness. Nonwoven materials are used in traditional wound healing approaches and have also been studied for the development of advanced wound care materials that offer both functionality and innovation in wound healing and tissue engineering applications. For example, various materials have been developed to provide wound dressings incorporating bioactive molecules, inorganic materials, and/or antimicrobial treatments to assist in dermal wound healing. Such modification of nonwoven structures is generally performed by incorporating the active material during the fiber production process, or as a post-processing treatment after structural formation of the nonwoven has been achieved.

Nonwoven materials have shown particular application in the area of controlled drug delivery (e.g., for wound healing and tissue engineering applications), where it is important to maintain a therapeutic drug level for prolonged periods of time. There is particular interest in development of biodegradable and biocompatible fibers that release specific drugs. Conventional delivery of a drug in successive doses leads to a high concentration of the drug in blood or tissue that varies over the duration of therapy. Therefore, over the duration of delivery, the concentrations may go over the maximum value (Cₘₐₓ), leading to risk of biotoxicity, or fall below the minimum effective concentration (Cₘᵢₙ), limiting the therapeutic effect. In order to obtain the highest therapeutic level, an optimum concentration C (Cₘᵢₙ<C<Cₘₐₓ) should be maintained in the tissue throughout the duration of healing. Controlled delivery techniques design the bioavailability of the drug to be close to this optimum value during therapy. In a controlled release mode, a lower amount of drug needs to be delivered and thus minimizes potential side effects.

When designing a polymer scaffold for a specific drug delivery application, it is desirable to incorporate drugs into the polymers that will be released during the growth of the tissue. Nanofibrous mats, having a porous structure, have great potential for use as the polymeric template in drug delivery applications. The release of the drug from such polymeric fibers may occur via a variety of mechanisms: 1) diffusive transfer through the polymer matrix to the surrounding tissue; 2) release of the dissolved or suspended drug due to slow biodegradation or erosion of the surface layers of the fiber; 3) slow release of covalently bonded drug via hydrolytic cleavage of the linkages; or 4) rapid delivery of the drug due to dissolution of the fiber.

Nonwoven materials are also studied for their potential application as scaffolds in tissue engineering. An important objective of tissue engineering is to provide an alternative for conventional transplants by developing three-dimensional polymeric scaffolds seeded with cells. Live tissue consists of collections of cells arranged in complex geometries within an extracellular matrix (ECM) that provides structural support to the cells. It is known that the fibrillar structure of collagen within the ECM is important for cell attachment, proliferation, and differentiation function in tissue cultures. Nanofibrous mats, by mimicking the morphological characteristics of collagen, may lead to engineered tissue more closely resembling native tissues.

In particular, electrospun nanofibers are one type of material that are of considerable interest in healthcare due to their high surface area, porous structure, and absorbent properties. The technique of electro spinning offers an inexpensive, yet reproducible method to create fibers on the submicron scale, and provides a material with inherent similarities to the natural extracellular matrix (ECM) surrounding cells and tissues in vivo. Electrospun nanofibers can be tailored to achieve fibers with compositions, as well as different structures and morphologies. See, e.g., Li et al., J. Biomed. Mater. Res. 2002, 60(4): 613-621, which is incorporated herein by reference. However, the use of electrospun materials has been limited due to low production rate and the inability to control release of compounds loaded therein. For example, the main mechanism to release an embedded compound is generally for the fibrous matrix to degrade (e.g., by surface erosion of bulk degradation) within a fluidic environment. As a result, the release properties of a particular compound are extremely hard to control, as is the responsive dose systemically administered to a potential patient.

It would be desirable to provide electrospun fibrous materials capable of functioning as scaffolds for various biological applications.

### SUMMARY OF THE INVENTION

The present invention provides electrospun fibrous materials that find application in a wide range of applications, particularly in the field of healthcare. In certain embodiments, fibrous materials are prepared by electro spinning and can optionally be functionalized on the interior and/or exterior, e.g., with therapeutic agents. The type and makeup of the fibrous materials can vary. For example, the present disclosure provides single polymeric component fibers, core/sheath fibers, hollow fibers, porous fibers, and combinations thereof, which can optionally be functionalized. Exemplary types of therapeutic agents that can be incorporated within or otherwise associated with the fibers include, but are not limited to, silver-containing compounds (e.g., silver nanoparticles, silver microparticles, silver-containing polymers, etc.), non-steroidal anti-inflammatory drugs, and calcium containing compounds to name but a few of many potential therapeutic agents.

In one aspect of the invention is provided a fibrous web comprising a plurality of electrospun hollow, porous fibers comprising one or more biocompatible polymers, wherein the fibers further comprise one or more therapeutic agents contained within the hollow portion of the fibers, coated on the surfaces of the hollow, porous fibers, or both contained within the hollow portion of the fibers and coated on the surfaces of the fibers. In certain embodiments, one therapeutic agent can be contained within the hollow portion of the fibers and a second therapeutic agent can be coated on the surfaces of the hollow, porous fibers.

In another aspect of the invention is provided a fibrous web comprising a plurality of electrospun fibers having one or more therapeutic agents imbedded in the fibers, coated on the surfaces of the fibers, or both imbedded in the fibers and coated on the surfaces of the fibers, wherein the one or more therapeutic agents comprise a silver-containing therapeutic agent. The electrospun fibers can have a form, for example, selected from the group consisting of single-component fiber, core/sheath fiber, hollow fiber, porous fiber, and combinations thereof.

In some embodiments, the silver-containing therapeutic agent comprises silver nanoparticles, and in some embodiments, the silver-containing therapeutic agent comprises silver microparticles. Silver microparticles can have average diameters, for example, of between about 1 micron and about 10 microns and/or can have average surface areas of at least about 2 m²/g. Silver nanoparticles and/or silver microparticles can, in some specific embodiments, be imbedded within the fibers.

In a further aspect of the invention is provided a fibrous web comprising a plurality of electrospun fibers, wherein the plurality of electrospun fibers comprise one or more polymers and one or more cellulosic materials imbedded in the fibers, coated on the surfaces of the fibers, or both imbedded in the fibers and coated on the surfaces of the fibers. The cellulosic material can, for example, comprise cotton. The amount of cellulosic material incorporated within the web can vary; for example, in some embodiments, the cellulosic materials can be present in an amount of between about 5% and about 40% by weight of the fiber. In certain embodiments, such cellulosic material-containing webs can further comprise one or more therapeutic agents as described herein.

In one particular embodiment, the invention provides a fibrous web comprising a plurality of electrospun fibers comprising one or more biocompatible polymers and at least one of: (i) one or more therapeutic agents imbedded in the fibers, coated on the surfaces of the fibers, or both imbedded in the fibers and coated on the surfaces of the fibers, wherein the one or more therapeutic agents comprise a silver-containing therapeutic agent; and (ii) one or more cellulosic materials imbedded in the fibers, coated on the surfaces of the fibers, or both imbedded in the fibers and coated on the surfaces of the fibers. The silver-containing therapeutic agent can be any silver-containing therapeutic agent disclosed herein and the cellulosic material can be any cellulosic material disclosed herein.

In certain embodiments of the present disclosure, the webs described herein comprise electrospun fibers comprising a polymer selected from the group consisting of polylactic acid (PLA), polycaprolactone (PCL), polyethylene oxide (PEO), polyvinyl alcohol (PVA), polyglycolic acid (PGA), poly(ethylene-co-vinylacetate) (EVA), poly(ethyleneimine) (PEI), poly(2-hydroxyethyl methacrylate) (pHEMA), poly(2-hydroxypropyl methacrylate), poly(2-(dimethylamino)ethyl methacrylate), polylysine, poly(methylmethacrylate) (PMMA), polypyrroles, cyclodextrin, poly(a-[4-aminobutyl]-1-glycolic acid) (PAGA), poly(2-(dimethylamino)ethyl methacrylate) (pDMAEMA), poly(enol-ketone) (PEK), N-(2-hydroxypropyl)methacrylamide (HPMA), and blends, derivatives, and copolymers thereof. In certain embodiments, the webs can comprise two or more therapeutic agents.

In another aspect of the invention is provided a bandage for wound treatment comprising the fibrous web of any embodiment described herein. In certain embodiments, such bandages can further comprise one or more additional layers of fibrous webs.

In a further aspect of the present invention is provided a method of producing a fibrous web of hollow, porous fibers comprising one or more therapeutic agents, comprising: coaxially electrospinning two polymer solutions to produce fibers having a core-sheath cross-sectional configuration; and selectively dissolving the core component of the fibers, wherein the volatility of the solvent and the temperature and humidity at Which the electrospinning is conducted are sufficient to provide pores on the exterior of the fibers. The solution can comprise, for example, dichloromethane.

In a still further aspect of the present invention is provided a method of producing a fibrous web of fibers having silver nanoparticles, silver microparticles, or both silver nanoparticles and silver microparticles imbedded in the fibers, comprising: electrospinning a polymer solutions comprising silver nanoparticles, silver microparticles, or both silver nanoparticles and silver microparticles to produce fibers having silver nanoparticles, silver microparticles, or both silver nanoparticles and silver microparticles imbedded therein. In some embodiments, the solution comprises dimethylformamide, chloroform, or a combination thereof. The polymer can comprise, for example, poly(lactic acid).

In an additional aspect of the invention is provided a method of producing a fibrous web of fibers having cellulosic materials incorporated therein, comprising: forming a polymer solution; adding cellulosic particles to the polymer solution; and electrospinning the polymer solution containing cellulosic particles to produce an electrospun web of fibers having cellulosic materials incorporated therein. In certain embodiments, the cellulosic particles are present in an amount of between about 5% and about 40% by weight of the fiber. The average dimensions of the cellulosic particles can vary. For example, in some embodiments, the cellulosic particles comprise particles with a largest average dimension of less than about 500 microns and in some embodiments, the cellulosic particles comprise particles with a largest average dimension of between about 200 and about 400 microns.

### BRIEF DESCRIPTION OF THE DRAWINGS

In order to provide an understanding of embodiments of the invention, reference is made to the appended drawings, which are exemplary only and should not be construed as limiting the invention.

FIG. 1 is a schematic of a general electrospinning setup;

FIG. 2 is a schematic of a co-axial electrospinning setup;

FIGS. 3a), 3b), and 3c) are depictions of core-sheath fibers, porous fibers, and porous core-sheath fibers (or porous hollow fibers), respectively;

FIG. 4 is a scanning electron microscopy (SEM) image of a bead in core-sheath scaffold loaded with 6 weight percent of silver nanoparticles, showing full encapsulation;

FIG. 5 is a comparative photograph of an electrospun PLA fiber exposed to water and an electrospun cotton-loaded PLA fiber exposed to water;

FIGS. 6a) and 6b) are SEM micrographs of core-sheath with tricalcium phosphate (TCP) (a) and single component with TCP (b) fibers;

FIGS. 7a)-7c) are SEM micrographs of porous PLA fibers;

FIGS. 8a)-8f) are SEM micrographs of porous PLA scaffolds at 5 wt % TCP (a, b, c) and 10 wt % TCP (d, e, f);

FIG. 9 is a graph of in vitro nanofiber weight loss for different fiber structures;

FIG. 10 is a graph of release profiles of different fiber structures;

FIGS. 11a)-11d) are SEM images of single component nanofibers with 2 weight percent silver nanoparticles (a, b) and 5 weight percent silver (c, d);

FIGS. 12a)-12d) are SEM images of core-sheath nanofibers with 2 weight percent silver nanoparticles (a, b) and 5 weight percent silver (c, d);

FIGS. 13a) and 13b) are SEM micrographs of core-sheath nanofibers containing 2 wt % silver nanoparticles;

FIGS. 14a)-14d) are SEM micrographs of nanofibers of (a) 15 wt % PLA, 3 wt % Ibuprofen; (b) 15 wt % PLA, 6 wt % Ibuprofen; (c) 12 wt % PLA, 4 wt % Ibuprofen; and (d) 12 wt % PLA, 8 wt % Ibuprofen;

FIGS. 15a)-15f) provide a comparison of spunbonded fibers (a, d), electrospun component nanofibers (b, e), and electrospun porous fibers coated with Silvadur ET™ (c, f);

FIGS. 16a)-16d) are SEM micrographs of PLA fiber materials containing silver nanoparticles before (a, c) and after (b, d) release of the silver nanoparticles;

FIGS. 17a)-17d) are viability images of human adipose drive stem cells seeded on electrospun fibers;

FIGS. 18a) and 18b) are graphs, showing ibuprofen release a) at room temperature and b) at 37° C.;

FIG. 19 is fluorescent images of viability of cells on control (0%) and antimicrobial (31.25%) scaffolds on days 1, 4 and 7;

FIG. 20 is a graph showing AlamarBlue reduction for cells seeded on different scaffolds;

FIGS. 21a)-21o) are SEM micrographs of fibroblast cells seeded on PLA bandages (a, b, c) and PLA bandages coated with varying amounts of Silvadur ET (d-o);

FIGS. 22a)-22c) are SEM micrographs of porous fibers spun in 65% relative humidity (a), 75% relative humidity (b), and 85% relative humidity (c);

FIGS. 23a) and 23b) are SEM micrographs of porous fibers spun in 75% relative humidity (a) and 85% relative humidity (b);

FIG. 24 is a bar graph of human skin keratinocyte proliferation on ibuprofen-loaded scaffolds;

FIG. 25 is an SEM micrograph of silver microparticles;

FIG. 26a)-f) are SEM (a, b, d, and e) and TEM (c and f) images of PLA nanofibers containing silver nanoparticles (a, b, and c) and highly porous silver microparticles (d, e, and f);

FIG. 27 is a schematic of a co-culture system to evaluate human skin and/or other mammalian cells in combination with bacteria on 3-dimensional nanofibrous scaffolds;

FIG. 28 is a graph of release profiles of silver ions from PLA scaffolds loaded with silver nanoparticles and silver microparticles;

FIG. 29 is a graph of human epidermal keratinocyte proliferation on scaffolds without bacteria in culture medium, where different letters indicate significant differences (p value <0.05);

FIG. 30a)-i) are SEM micrographs showing the viability of human epidermal keratinocytes on scaffolds, with pure PLA (a, b, and c), PLA containing silver nanoparticles (d, e, and f) and PLA containing silver microparticles (g, h, and i) at day 1 (a, d, and g) and day 3 without bacteria (b, e, and h) and day 3 with bacteria (c, f, and i);

FIG. 31a)-f) are SEM micrographs of human epidermal keratinocytes seeded on scaffolds (a and d are pure PLA, b and e are silver nanoparticle-loaded PLA, c and f are silver microparticle-loaded PLA), taken in the absence of S. aureus (d, e, and f) and the presence of S. aureus at day 3 (a, b, and c);

FIG. 32 is a graph of human epidermal keratinocyte DNA quantitation on scaffolds both in the presence and absence of S. aureus bacteria, where different letters indicate significant differences (p value <0.05); and

FIG. 33 is a graph of S. aureus bacterial growth on a cellular or human epidermal keratinocyte-seeded scaffolds, where different letters indicate significant differences between groups (p value <0.05).

### DETAILED DESCRIPTION OF THE PREFERRED EMBODIMENTS

The present invention now will be described more fully hereinafter. This invention may, however, be embodied in many different forms and should not be construed as limited to the embodiments set forth herein; rather, these embodiments are provided so that this disclosure will be thorough and complete, and will fully convey the scope of the invention to those skilled in the art. As used in this specification and the claims, the singular forms “a,” “an,” and “the” include plural referents unless the context clearly dictates otherwise.

The present invention relates to biocompatible nonwoven materials with various applications in the healthcare industry. For example, the materials described herein can find particular use in controlled, localized drug delivery, tissue engineering, and wound healing applications. In certain aspects of the invention, such biocompatible nonwoven materials are electrospun nonwoven materials, which may exhibit unique morphologies and/or incorporate various therapeutic agents or other materials therein. In some embodiments, fibrous materials that are “smart release materials,” due to the design of fiber architecture and physical property of the constituent polymer(s) are provided.

Electrospinning utilizes the interplay between electrical forces and surface tension to create fibers by applying a strong electric field between a charged drop of polymer solution and a collection plate. Generally, the charged drop of polymer solution is ejected through a spinneret to form substantially continuous, ultrathin fibers, which can be collected on the collection plate in the form of a fibrous mat. Although the method as disclosed herein describes the formation of a “mat,” it is understood that collection surfaces of varying geometries can be used (e.g., the fibers can be collected on a rotating mandrel to form a tube-like structure).

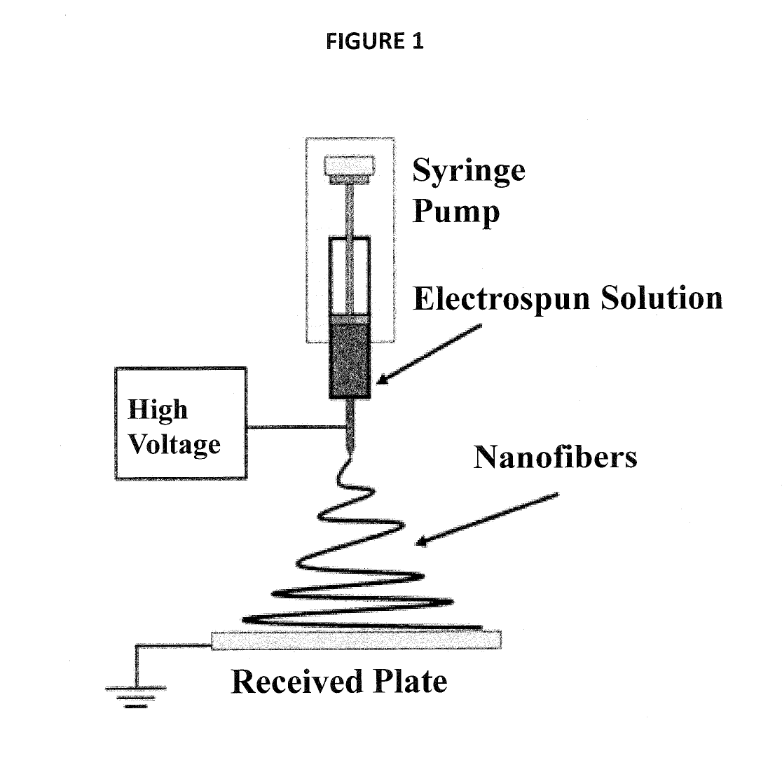

A typical electrospinning apparatus is shown in FIG. 1, which illustrates the deposition of nanofibers onto a collection plate from an electrically charged syringe pump. This electrospinning setup basically consists of a metallic needle attached to a syringe filled with polymer solution, a grounded collector, and a high voltage power supply connected between the needle and the collector charging them positively and negatively, respectively. The polymer solution generally contains the polymer or blend of polymers to be electrospun and one or more solvents. The solvent or solvents can be, for example, dichloromethane (DMC), ethylene acetate (EA), dichloroethylene (DCE), dimethylformamide (DMF), hexafluoroisopropanol (HIFP), dichloromethane (DCM), tetrahydrofuran (THF), ethyl acetate (EA), chloroform, acetone, heptane, isopropylalcohol, octanol and toluene, and water. The concentration of the polymer solution can vary and in some embodiments, the polymer solution can comprise about 5% to about 25% by weight polymer.

The feeding rate of the polymer solution can be controlled, for example, by a metering syringe pump. During electrospinning, the polymer solution is delivered to the needle at a constant feed rate and a pendant droplet of solution emerges at the tip of the needle. By increasing the voltage applied to the needle the charged droplet undergoes deformation into a conical shape known as a “Taylor cone.” As the strength of the applied voltage increases to the point at which the electrostatic forces overcome the surface tension of the solution, a fine jet of solution erupts from the droplet and moves toward the collector. The jet initially follows a straight path and then due to the repulsion of charges in the jet, it undergoes “bending instability.” During this phase, the jet is drawn, solvent evaporates, and fine fibers deposit onto the collector. The fine fibers can be deposited in an aligned fashion but are more commonly deposited in a random alignment (e.g., forming a random web of fibers).

Electrospinning can provide fibers with varying average diameters, including micro- and nano-meter diameters. For example, electrospun fibers as described herein are generally intended to include fibers having average outer diameters of between about 20 nm and about 5 μm, generally between about 200 nm and about 1.5 μm. The average outer diameter can, in certain embodiments, vary depending on the specific type and morphology of the fiber being produced. Average porosities of mats produced by electrospinning can vary, but are typically greater than about 80%. As such, these materials not only resemble the natural extracellular matrix in vivo, but also provide an extremely large surface area to volume ratio for maximizing the interaction of the fibers with a surrounding medium.

In various embodiments of the present disclosure, electrospun fibers having a range of morphologies are provided. For example, single component fibers, porous fibers, and core-sheath fibers are described in further detail herein. Varying morphologies and structures of electrospun fibers can greatly influence their potential for drug delivery and tissue engineering applications. A traditional electrospinning system, such as that depicted in FIG. 1 must, in some embodiments, be altered for the production of such varying morphologies.

There are a number of intrinsic and extrinsic factors in electrospinning that depend on the specific apparatus and polymer system. Intrinsic factors include, but are not limited to, solution parameters such as polymer concentration, solvent system, viscosity, and conductivity. For example, the polymer concentration and/or viscosity of the solution must be high enough to ensure a sufficient amount of chain entanglement, allowing for the formation of substantially continuous fibers rather than small droplets. Extrinsic factors include, but are not limited to, solution feed rate, electric field, and ambient parameters such as temperature and humidity. For example, temperature and humidity greatly affect fiber formation as they can alter solvent evaporation, leading to morphological changes and impeding fiber formation. Volumetric flow rate determines how large the pendant droplet becomes, and can alter Taylor cone formation. Electric field strength is crucial in overcoming the inherent surface tension of the polymer and must the necessary force required to produce a stable Taylor cone.

In some aspects of the invention, simple, non-porous electrospun fibers, having a substantially uniform cross-section are prepared using a single polymeric component. These and other electrospun fibers described herein can be doped with one or more additives (e.g., therapeutics), as discussed in detail below.

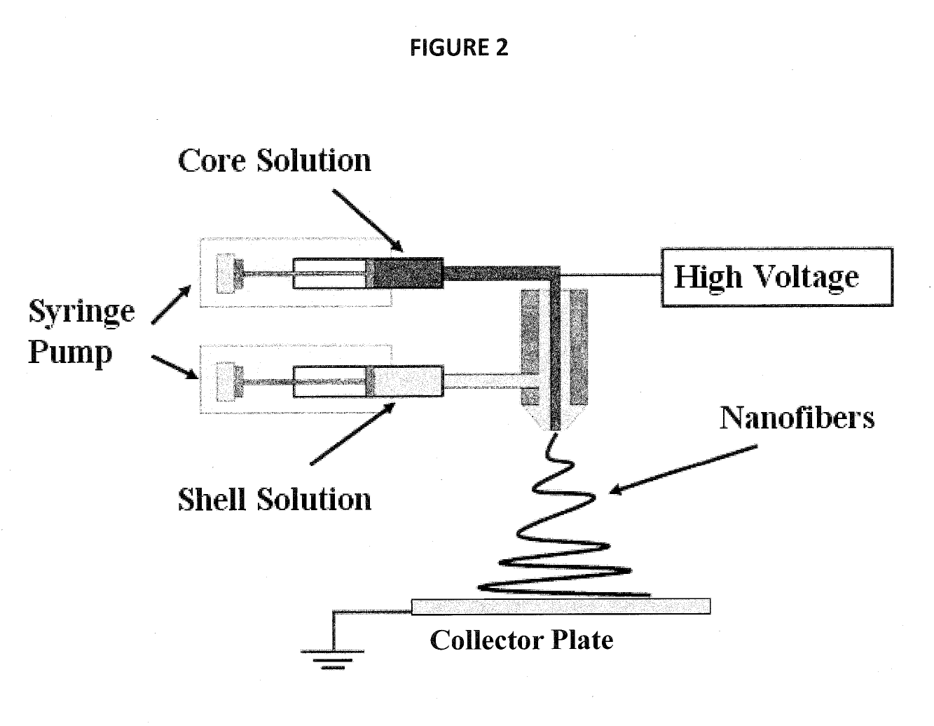

In certain aspects of the present invention, electrospun core-sheath fibers are provided. One exemplary method for co-axial electrospinning for the production of core-sheath nanofibers is similar to that used for spinning single component nanofibers except that a pair of capillaries that are co-axially aligned deliver two polymer solutions and are both charged simultaneously. Various systems have been designed; a simple set-up modified from the single capillary system is shown in FIG. 2. The same intrinsic and extrinsic factors described above for single component electrospinning apply to core-sheath electrospinning. Furthermore, since the sheath and the core solutions are in contact and undergo the same bending instability and whipping motion, the degree of dissimilarity between them, in terms of composition, and physical and rheological properties, plays an important role in the formation of the composite fiber. Co-axial electrospinning can be used to make a variety of different fiber structures. For example, this method can provide core-sheath bicomponent nanofibers, fibers from traditionally non-electrospinnable materials, hollow fibers, and fibers containing partially or fully encapsulated materials.

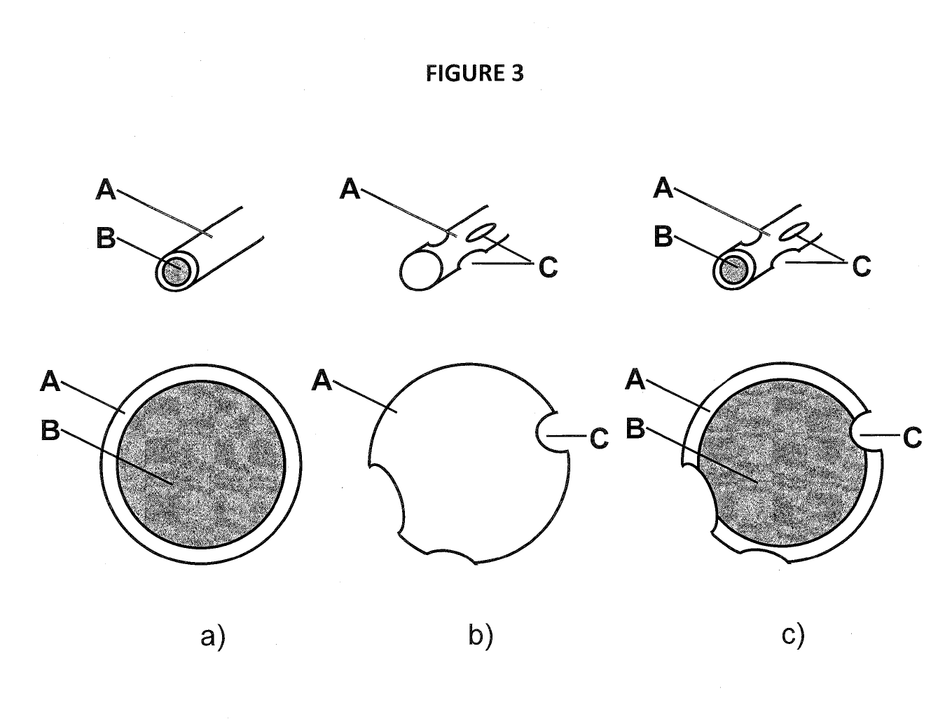

Exemplary core-sheath fibers are illustrated in FIG. 3a), wherein the top picture represents a side view of a portion of a cut fiber and the bottom picture represents a cross-section of a fiber. In certain embodiments, A is the sheath and B is the core. Any two immiscible polymers can be used as the components A and B. The polymers constituting these components can vary and may be synthetic or naturally derived. In preferred embodiments, one or more of the polymers are biocompatible and/or biodegradable. In certain exemplary embodiments, A and B are selected from the group consisting of polylactic acid (PLA), polycaprolactone (PCL), polyethylene oxide (PEO), polyvinyl alcohol (PVA), polyglycolic acid (PGA), poly(ethylene-co-vinylacetate) (EVA), poly(ethyleneimine) (PEI), poly(2-hydroxyethyl methacrylate) (pHEMA), poly(2-hydroxypropyl methacrylate), poly(2-(dimethylamino)ethyl methacrylate), polylysine, poly(methylmethacrylate) (PMMA), polypyrroles, cyclodextrin, poly(a-[4-aminobutyl]-1-glycolic acid) (PAGA), poly(2-(dimethylamino)ethyl methacrylate) (pDMAEMA), poly(enol-ketone) (PEK), N-(2-hydroxypropyl)methacrylamide (HPMA), and blends, derivatives, and copolymers thereof.

In other embodiments, component B of FIG. 3a) represents an empty core, such that the fibers are hollow fibers. Such fibers can be prepared as described above and treating the fibers as to remove the interior component (e.g., by dissolving the interior component). Appropriate selection of the core and sheath components can allow for selective dissolution of the core component while maintaining the sheath component. For example, in specific embodiments, PLA or PCL can be used as the sheath component A and PVA can be used as the core component B. In such embodiments, the core-sheath fibers are produced as provided herein and the fibers can then be placed in water, which dissolves the PVA portion. As PLA and PCL are not soluble in water, the exterior sheath component A is maintained and, as the PVA has been removed by dissolution, the resulting fibers are hollow PLA or PCL fibers.

Porous fibers may be advantageous for certain applications and can, in some embodiment, provide high specific surface area together with high porosity. Such fibers may be used in many applications such as cell growth and proliferation, carriers for encapsulating therapeutic agents, and biological filtering media. Certain porous electrospun fibers and methods for producing them are described, for example, in U.S. Patent Application Publication No. 2012/0015020 to Pourdeyhimi et al., which is incorporated herein by reference. Exemplary single-component porous fibers are illustrated in FIG. 3b), wherein the top picture represents a side view of a portion of a cut fiber and the bottom picture represents a cross-section of a fiber. A represents the polymeric material comprising the fiber, and C represents the pores on the surface of the fibers.

Although other methods are known for providing pores in a fibrous structure, in certain embodiments, porous fibers are provided by incorporating a highly volatile solvent in an environment with a specific temperature and humidity to make porous fibers in a one step process. In this approach, pores form due to the fast evaporation of solvent, which leads to evaporative cooling of the surface of the jet during electrospinning. The solvent can vary; one exemplary volatile solvent that provides for pore formation is dichloromethane. A significant temperature difference between the surface of the fibers and the surrounding media also aids the process. As a result of these phenomena, the nucleation and growth of moisture in the ambient media occurs resulting in condensation and growth of moisture in the form of droplets. As the jet dries and the solvent evaporates, pores form on the surface of the fibers. In certain embodiments, this method gives rise to fibers having relatively uniform diameters and surface pores with substantially consistent dimensions. A schematic drawing of a side view (top) and a cross-sectional view (bottom) of a porous, core-sheath or hollow fiber is provided in FIG. 3b).

Advantageously, according to certain embodiments of the invention, these features can be provided in a single type of fiber: a porous, hollow fiber. These fibers can be provided by combining the methods described herein, e.g., by selecting appropriate core and sheath components to allow for selective dissolution of the core, electro spinning the core and sheath components from a volatile solvent in an environment having a given temperature and humidity level to provide a porous core-sheath fiber, and then dissolving the core component to give a porous, hollow fiber.

Various additional materials can be incorporated within the fibers described herein (e.g., single-component fibers, core-sheath fibers, hollow fibers, porous fibers, porous core-sheath fibers, and porous hollow fibers). In some embodiments, the present application provides a fibrous web comprising porous nanofibers and one or more therapeutic agents incorporated therein.

For example, in some embodiments, one or more therapeutic agents can be incorporated within a fibrous structure as described herein. The type of types of therapeutic agents added can vary and may be, for example, pharmaceuticals, dietary supplements (e.g., nutraceuticals), or biomolecules (e.g., human stem cells). Exemplary classes of pharmaceutical agents include, but are not limited to, antimicrobials (e.g., antibacterial agents, including silver and silver-containing compounds, tetracycline hydrochloride, and rifampicin; antiviral agents; antiparasitic agents, and antifungal agents); anti-inflammatories/analgesics (e.g., steroids, including dexamethasone, and non-steroidal anti-inflammatory drugs (NSAIDS), including ibuprofen, salicylates, aspirin, acetaminophen, naproxen, morphine, opium); growth factors (e.g., neurotrophins, fibroblast growth factors, transforming growth factors, glial derived growth factors); polysaccharides (e.g., hyaluronan); proteins (e.g., bovine serum albumin), collagen, oligonucleotides (e.g., DNA, RNA, cDNA, PNA, genomic DNA, and synthetic oligonucleotides), cells (e.g., mammalian cells), and minerals (e.g., tricalcium phosphate, TCP). Other therapeutic agents that can be used according to the invention include those types of active agents disclosed in U.S. Patent Application Publication No. 2012/0015020 to Pourdeyhimi et al., which is incorporated herein by reference.

The rate of release of such therapeutic agents can be controlled. For example, the rate of release can vary depending on the location of the therapeutic agent in relation to the fibrous structure. In some embodiments, the therapeutic agent is within a pore or cavity of a fiber (e.g., in pores on the surface of a fiber or within a hollow core of a hollow fiber). In some embodiments, the therapeutic agent is incorporated within a solid component of a fiber (e.g., incorporated directly into the core or sheath component). In some embodiments, a therapeutic agent can be coated onto the exterior of the fiber. In further embodiments, two or more of these approaches can be combined. For example, one therapeutic agent can be incorporated within the core component of a porous, core-sheath fiber and another therapeutic agent can be incorporated within the exterior pores of the fibers.

In some embodiments, controlled, consistent release can be obtained using the materials provided herein. In some embodiments, a relatively fast burst release can be provided. In certain embodiments, these features are combined such that a relatively fast burst release of one therapeutic agent is provided, along with extended release of a second therapeutic agent (which may be the same or different than the first therapeutic agent). The rate of release can, for example, be controlled to some extent by incorporating the therapeutic agents at different locations within the fibrous structure (e.g., within the sheath or core component or both the sheath and core components of a sheathcore fiber). In certain embodiments, therapeutic agents located within the core component of a core-sheath fiber and therapeutic agents located within the hollow core of a hollow fiber may exhibit slower release than therapeutic agents incorporated within a sheath component, located within external pores, or coated on the exterior of a fiber. The rate of release can, in some embodiments, be controlled by varying the polymer concentration, molecular weight, and morphology of the sheath layer of a hollow fiber to alter the polymer degradation.

One exemplary subclass of antibacterial compounds that can advantageously be incorporated within the structures described herein is silver-containing compounds. Historically, silver has been widely used in medicine as a topical treatment for burns, minor wounds and infections. Recently, it has attracted much interest from both the scientific and industrial textile communities as it can be used as a means of protecting textile products and their users from the negative impact of microbial contamination. Silver is relatively safe, effective, long lasting, and versatile and is highly toxic to a wide range of microorganisms. However, unlike some other available antimicrobial chemicals, it is not dangerous to humans when delivered in the proper chemical form and concentration. It is an FDA approved broad-spectrum biocide that kills over 650 disease-causing bacteria, fungi, viruses, and mold. There is no life-threatening risk caused by inhalation, ingestion, or dermal application. If silver penetrates into the human body, it goes into systemic circulation as a protein complex, and can be eliminated by the liver and kidneys.

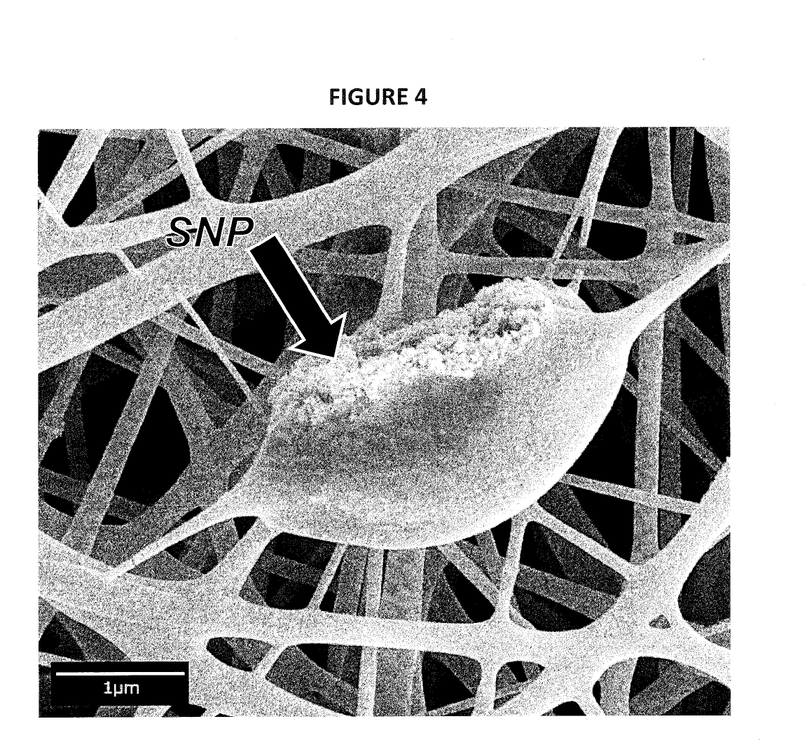

For example, silver nanoparticles can be used within the fibrous structures described herein. Although they can simply be incorporated within the fibrous structure, in some embodiments, silver nanoparticles are encapsulated within the fibers, as illustrated in FIG. 4. Silver nanoparticles can be incorporated within existing manufacturing processes and can be applied to any textile product as a fiber or fabric coating or by incorporation into the polymer prior to fiber extrusion. Silver nanoparticles might provide textiles with a long lasting, durable antimicrobial effect. Their high surface area to volume ratio allows for the achievement of excellent antimicrobial effects at low concentrations because more silver atoms on the surface of the nanoparticles are exposed toward the microbes. In addition, there is no need for specialized equipment as silver nanoparticles can be easily incorporated within existing manufacturing processes. Because of its resistance to high temperatures, silver can be applied to any textile product, not only as a fiber or fabric coating, but also by incorporating it into polymers before fiber extrusion.

Another exemplary silver compound is SILVADUR™ ET (Dow Chemical Company), which contains a proprietary polymer and silver in a water/ethanol solution and can be diluted in water. It is completely soluble in textile treatment baths, but forms an insoluble interpenetrating polymer network with Ag⁺ upon application to a fabric and drying. The formation of this insoluble network results in a very durable antimicrobial finish. When organisms land on the surface of the treated fabric, the free Ag⁺ interacts with the organism resulting in cell death. As the initial available Ag⁺ is diminished by interaction with organisms, more Ag⁺ is released from the complex and the process continues.

In certain embodiments, a silver material comprising silver microparticles is employed as a therapeutic agent. The microparticles can advantageously be in highly porous form (e.g., having an average surface area of at least about 1 m²/g, at least about 2 m²/g, or at least about 3 m²/g, e.g., with an average surface area of between about 1 m²/g and about 6 m²/g, between about 2 m²/g and about 5 m²/g, or between about 3 m²/g and about 4.5 m²/g). In some embodiments, the silver microparticles comprise agglomerations of silver nanoparticles and the microparticles may accordingly comprise nano-size structure/patterning on the surface thereof. The average diameter of the silver microparticles can vary and may be, for example, between about 0.1 micron and about 100 microns. In certain embodiments, the average diameter of the silver microparticles can be at least about 1 micron, at least about 5 microns, or at least about 8 microns, e.g., between about 1 and about 10 microns or between about 5 and about 10 microns. One exemplary commercially available silver microparticle that can be used in the present disclosure is available under the tradename MICROSILVER BG™ from BioGate Company, Nuremberg, Germany.

In certain embodiments, the rate of silver ion release from silver microparticle-loaded scaffolds is higher than that from silver nanoparticle-loaded scaffolds. Although not intending to be limited by theory, it is believed that, in some embodiments, the rate difference may be influenced by the location of the therapeutic agent within the fiber cross-section. For example, therapeutic agents located closer to the exterior surface of a fiber can be released faster than those located closer to the core of a fiber. Silver microparticles, in some embodiments, are incorporated within fibers as described herein to provide fibers comprising silver microparticles located at or near the exterior surface of the fibers. In contrast, silver nanoparticles can, in some embodiments, be incorporated within fibers to give fibers comprising silver nanoparticles incorporated closer to the core of the fiber cross-section. In some embodiments, the cytotoxicity and antimicrobial efficiency of fibers comprising silver microparticles are comparable to those of fibers comprising silver nanoparticles.

In some embodiments, ibuprofen is incorporated within the fibrous structures of the present application. Ibuprofen is a non-steroidal anti-inflammatory drug (NSAID) used to relieve pain and reduce inflammation, e.g., in a wound bed. In some embodiments, tricalcium phosphate (TCP), which induces and accelerates osteogenic differentiation of human stem cells, is incorporated in the structure of electrospun fibers. For further information on the role of TCP, see McCullen et al., Biomed. Mater. 2009 June; 4(2): 035002, which is incorporated herein by reference.

Various types of therapeutic agents can be incorporated within the fibrous structures described herein. The rate of release of the therapeutic agents from the fibrous structures can vary. In certain embodiments, the release profile of core-sheath fibers results in a relatively steady therapeutic agent release behavior while porous fibers typically result in a relatively high initial burst release of therapeutic agent. Although not intending to be limited by theory, it is believed that release from porous fibers is primarily due to diffusion, while release from core-sheath fibers is mostly due to bulk degradation.

These features can be combined and tailored to provide fibrous materials specifically targeted for certain applications. It has been found that different fiber morphologies are preferred for different applications. For example, by combining the typically high absorption/fast therapeutic agent release properties of a porous sheath with the typically sustained therapeutic agent release of a solid core, bandages can be provided that provide a longer duration of antimicrobial protection to the wound site while simultaneously promoting a drier wound site. Bandages should need to be used less frequently and, in particular, with the presently disclosed materials, old bandages in some embodiments will not need to be removed as they will degrade in the wound site in a biocompatible fashion while continuously delivering therapeutic agents (e.g., antimicrobial and potentially analgesic and/or anti-inflammatory compounds). In tissue engineering applications, where electrospun fiber scaffolds are used, regular nanofibers may be preferred.

In other embodiments, materials other than (or in addition to) therapeutic agents can be incorporated within the fibrous materials described herein. For example, in some embodiments, one or more natural materials (e.g., cellulosic materials such as cotton or Lyocell) can be incorporated. The form of the one or more natural materials can vary and may be in the form of a fiber, but more advantageously is in the form of smaller fragments.

In one embodiment, cotton is incorporated within a fibrous electrospun material in the form of a cotton powder. The powder can, in some embodiments, be present within the fiber structure itself (e.g., by incorporating the powder within the electrospinning solution such that as the fiber is forming, the powder is drawn out of the spinneret along with the polymer solution). The percentage of cellulosic material (e.g., cotton) to polymer (by weight) can be, for example, about 10%, about 20%, about 30%, or greater. For example, in some embodiments, the cellulosic material is present in an amount of between about 5% and about 40% by weight of the fiber. The cellulosic powder is generally in particulate form and the size and shape of the particles comprising the powder can vary. For example, in some embodiments, the cellulosic powder may comprise particles of substantially spherical shape or particles of an elongated (e.g., fibrous) form. The largest average dimensions of the particles can be, for example, less than about 500 microns or less than about 400 microns (e.g., between about 100 and about 500 microns, preferably between about 200 and about 400 microns). In some embodiments, the addition of cotton powder to the electrospinning solution can provide fibers having cotton fragments contained therein.

In further embodiments, other forms of cellulose can be used, e.g., pulp, pulverized cotton, or Lyocell or rayon fibers. In certain embodiments, Lyocell can be incorporated within a fibrous electrospun material. Lyocell and rayon are regenerated cellulose fibers made from dissolving bleached wood pulp, and which can be easily incorporated in the techniques described herein. This cellulosic fiber does not have the dirt removal problem that can exist with other fibers. One distinct advantage of these types of fibers is that they have a higher tendency to fibrillation as compared with other cellulosic fibers, due to a higher degree of crystallinity (which makes it easier to form particles).

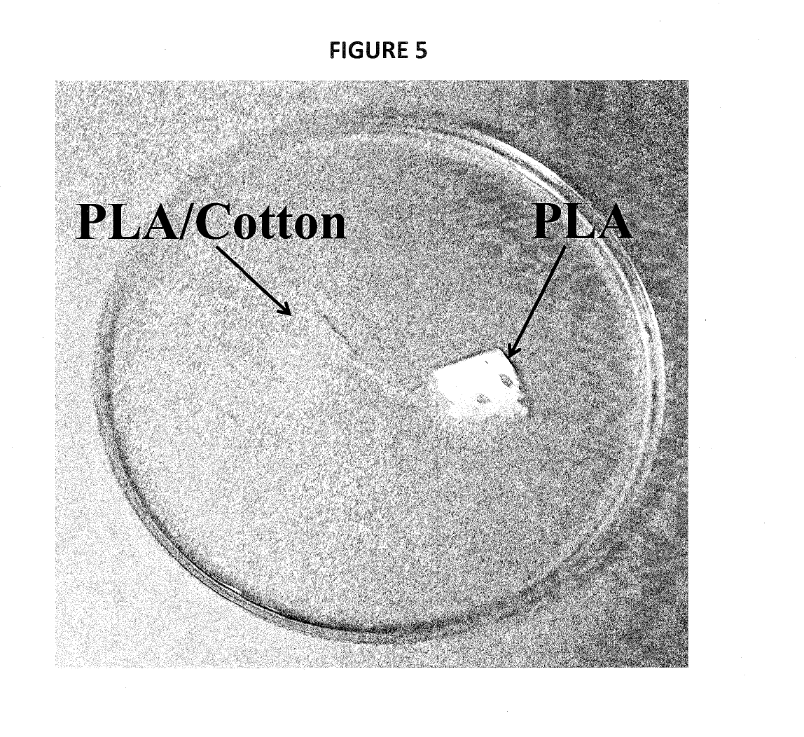

In certain embodiments, the incorporation of a natural material such as cellulosic material (e.g., cotton) within a fibrous structure as described herein can endow the structure with desirable properties. Particularly, the addition of cotton can increase the hydrophilicity of a polyester (e.g., PLA or PCL) fibrous mat. Increased hydrophilicity may be beneficial, e.g., in keeping a wound bed dry (thus assisting with inhibition of bacterial growth). The degree of hydrophilicity can be controlled by controlling the amount of cellulosic material (e.g., cotton) incorporated within the material. Thus, cellulosic particle (e.g., cotton)-containing fibrous structures may in some embodiments be useful in wound healing applications, for example, where the fibrous structure is designed so as to resemble desired morphological characteristics of native ECM surrounding cells in vivo and the presence of the cellulosic material serves to keep the wound bed dry. A comparative image of a PLA material and a PLA material incorporating 30% cotton are provided in FIG. 5, illustrating the increased hydrophilicity/wettability of the PLA/cotton nanofibers.

In some embodiments, fibrous materials described herein can be combined, e.g., in a multi-layered form. For example, in some embodiments, one or more layers of cellulosic material-containing (e.g., cotton-containing) fibrous webs are combined with one or more layers of single-component, single-component porous, core-sheath, porous core-sheath, hollow, or porous hollow fibers. In some embodiments, one or both of the layers further comprise one or more therapeutic agents as described herein. In some embodiments, electrospun materials according to the invention can be combined with other types of fibers, e.g., non-electrospun fibers. The fibers can be combined within the same web or can be provided in a layered structure.

The types of fibrous materials described in the present disclosure can be used in a wide range of end products, including, but not limited to, products for use in the medical field. For example, in certain embodiments, the fibrous webs described herein can be incorporated within such materials as wound dressings (e.g., bandages), wipes (e.g., sterile wipes), and implantable devices, including, but not limited to, tissue engineering scaffolds, drug delivery scaffolds, and patches. In still further embodiments, the fibrous materials described herein can be employed as layers and/or coatings on various types of devices.

Aspects of the present invention are more fully illustrated by the following examples, which are set forth to illustrate certain aspects of the present invention and are not to be construed as limiting thereof.

### EXPERIMENTAL

### Example 1

### Comparison of Antimicrobial Agents

A minimum inhibitory concentration (MIC) test was conducted to measure the efficiency of certain silver-based antimicrobials against each bacterial isolate. A sterile round-bottom plastic 96-well plate containing 100 ml of serially 1:2 diluted concentrations of antimicrobial solutions was inoculated with 100 ml of 5-8×105 CFU/ml of each bacterial isolate. Each antimicrobial sample was tested at 15 serially diluted concentrations starting at their original highest concentration (0.015-1000 μg/ml).

After the microplates were incubated for 24 h, the MIC was recorded to be the lowest concentration of antimicrobial which exhibited no visible growth. After 24 h of incubation, 10 μl of the suspension from all of the clear wells (showing no bacterial growth) was dropped onto a MH agar plate and incubated for 24 h. The minimum bactericidal concentration (MBC) was determined by the concentration that failed to kill bacteria.

The silver nanoparticles used had a spherical morphology with mean diameter of 20 nm (Nanocomposix, USA) and atomic molarity of 9.25 mM. The mass concentration of these nanoparticles dispersed in water was 1.0 mg/mL The Silvadur ET solution containing 2.6-3.1 wt % silver ions was used at its original concentration (1.0 mg/mL). Silver nitrate was also dissolved in distilled water to create a 1 mg/mL solution. A gram negative (Escherichia coli J53) and gram positive (staphylococcus aureus—a common bacteria to cause skin infections) bacteria were employed for the experiment. Cation-adjusted Mueller-Hinton (MH) broth and agar (Difco Laboratories, Detroit, Mich., USA) were utilized to prepare bacterial cultivating medium. Isolated bacterial colonies were grown overnight in an incubator (37° C., 5% CO₂) from frozen samples on an agar plate. For Agresistant bacteria (E. coli J53 [pMG101]), 100 mg/ml ampicillin sodium salt (Fisher Scientific, USA) was also added to the agar plates. The bacterial colony was suspended in phosphate buffered saline (PBS) to obtain 0.5 McFarland (105 CFU/ml). Microplates were incubated at 37° C. and shaken at 200 rpm for 24 hours. To control the accuracy of bacterial seeding density, bacteria were diluted in PBS at 103, 104, 105, and 106 and cultured overnight.

Minimum inhibition concentrations (Table 1) and minimum bactericidal concentrations (MBC; Table 2) were calculated for the silver nanoparticles, Silvadur ET solution, and AgNO₃ on each bacterial strain (n=6 per treatment condition).

TABLE 1
Values of Minimum Inhibitory Concentrations (MIC)

| | Staphylococcus | | |
|---|---|---|---|
| | aureus | Escherichia | E. Coli |
| Silver Type | (μg/ml) | coli J53 | J53pMG101 |
| Silver nanoparticles | 125 | 62.5 | — |
| Silvadur ET | 0.24 | 0.12 | 0.98 |
| Silver nitrate | 15.62 | 7.81 | — |

TABLE 2
Values of Minimum Bacterial Concentrations (MBC)

| | Staphylococcus | | |
|---|---|---|---|
| | aureus | Escherichia | E. Coli |
| Silver Type | (μg/ml) | coli J53 | J53pMG101 |
| Silver nanoparticles | 250 | 125 | — |
| Silvadur ET | 0.98 | 0.24 | 3.91 |
| Silver nitrate | 31.25 | 15.62 | — |

The results of this study indicated that the Silvadur ET solution was significantly more efficient in inhibiting bacterial growth in comparison with silver nanoparticles. Moreover, more antimicrobials were required to inhibit the growth of S. aureus bacteria relative to E. coli bacteria. Plasmid pMG101 is a silver resistant plasmid containing nine genes that also confers resistance to mercury, tellurite and several antibiotics. As bacterial contact with Ag increases, the number of Ag-resistant bacteria will correspondingly increase. Therefore, silver resistant bacteria have been reported in Ag-saturated environments. Our observation of silver resistant E. coli J53 (pMG101) in the presence of silver nanoparticles and silver nitrate confirmed the resistance of E. coli J53 to silver. However, results from innoculating Silvadur ET solution with this bacterium suggests that there are additional components other than silver ions in the Silvadur ET solution that are responsible for killing this strain of bacteria.

### Example 2

### Incorporation of Therapeutic Agents into Scaffolds of Varying Fiber Architectures

Different polymers, solution concentrations and solvent combinations were used in this study. In each case the accurate weight percentage of polymer was weighed and dissolved in the appropriate solvent. The mixture was stirred on a magnetic stirrer plate for at least 12 hours until a homogeneous solution was obtained. When homogeneous solutions could not be obtained at room temperature, the mixtures were heated on a heated magnetic stirrer. Polymer solutions were used within 24 hours of preparation to eliminate evaporative loss of solvent and consequent change in solution concentration. Table 3 shows the polymers used in the following experiments.

TABLE 3
Polymers used in Example 2

| Polymer | Abbreviation | Molecular weight |
|---|---|---|
| Polylactic acid | PLA | 70,000 g/mol |
| Polycaprolactone | PCL | 80,000 g/mol |
| Polyvinyl alcohol | PVA | 100,000 g/mol |
| Polyethylene oxide | PEO | 400,000 g/mol |

### Tricalcium Phosphate (TCP)

Tricalcium phosphate is a calcium salt of phosphoric acid with the chemical formula Ca₃(PO₄)₂. Calcium phosphate is the primary component of mineral in bone. TCP, a soluble form of calcium phosphate, can degrade in vitro, releasing Ca²⁺ and PO₃⁻⁴ into the surrounding environment. Extra pure TCP in the form of spherical crystals with average particle size of 40 nm (Sigma, St Louis, Mo.) was used. Table 4 sets forth the electrospinning parameters utilized to form nanofibers containing TCP.

TABLE 4
Electrospinning Parameters for Nanofibers with TCP

| Spinning Parameters | TCP in core | TCP in sheath |
|---|---|---|
| Voltage (kV) | 14 | 14 |
| Spinning distance (cm) | 15 | 15 |
| Core feed rate (μm/min) | 0.6 | 0.4 |
| Sheath feed rate (μm/min) | 1.2 | 1.2 |

In order to investigate the effect of fiber structures on the release profile of drugs, tricalcium phosphate was used as a model drug and was incorporated within PLA polymer matrices comprising single component, core-sheath and porous fiber forms. Tricalcium phosphate was incorporated in the fibers using both single component and coaxial electrospinning systems. Single-component and core-sheath fiber scaffolds were electrospun using 12% PLA solution in chloroform:DMF (3:1) containing 10% TCP. For core-sheath fibers, both the sheath component and the core component comprised PLA and the TCP was included in either the core or the sheath component. Porous fibers were successfully created from 12% PLA solution in dichloromethane (in the absence of TCP). TCP-containing, porous PLA fiber scaffolds were then prepared by incorporating 5 wt % and 10 wt % TCP into the spinning solutions.

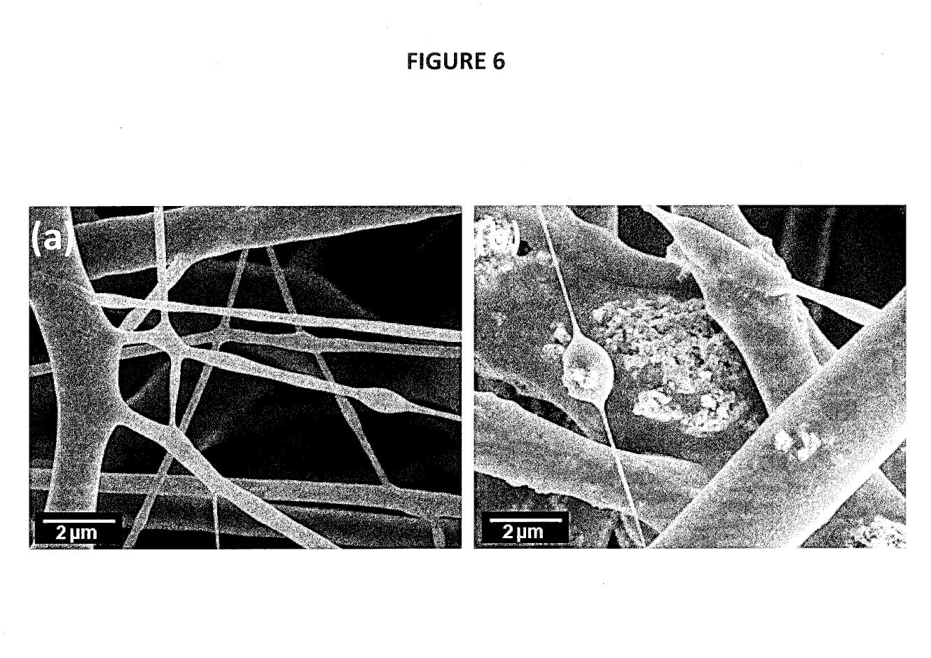

Analysis of the surface morphologies of these fiber structures indicated that use of coaxial electrospinning (with TCP located in the core component) inhibited TCP particle formation on the surfaces of the fibers, resulting in fibers with smooth surfaces (and TCP isolated in the core). In contrast, single component fibers exhibited TCP both on the interior of the fibers and on their surfaces, as indicated by their rougher surface morphology. FIG. 6 shows scanning electron microscopy (SEM) micrographs of core-sheath fibers comprising TCP in the core component (a) and single component fibers comprising TCP (b).

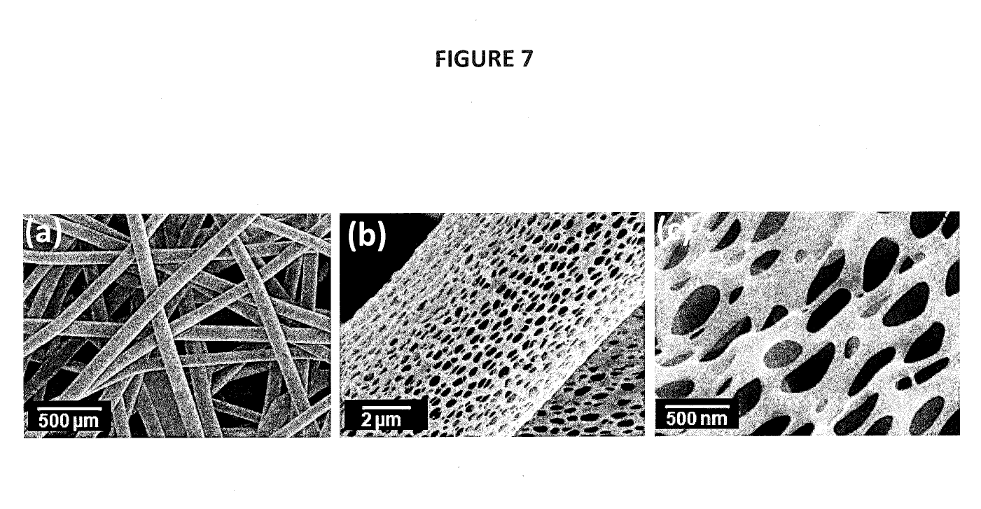

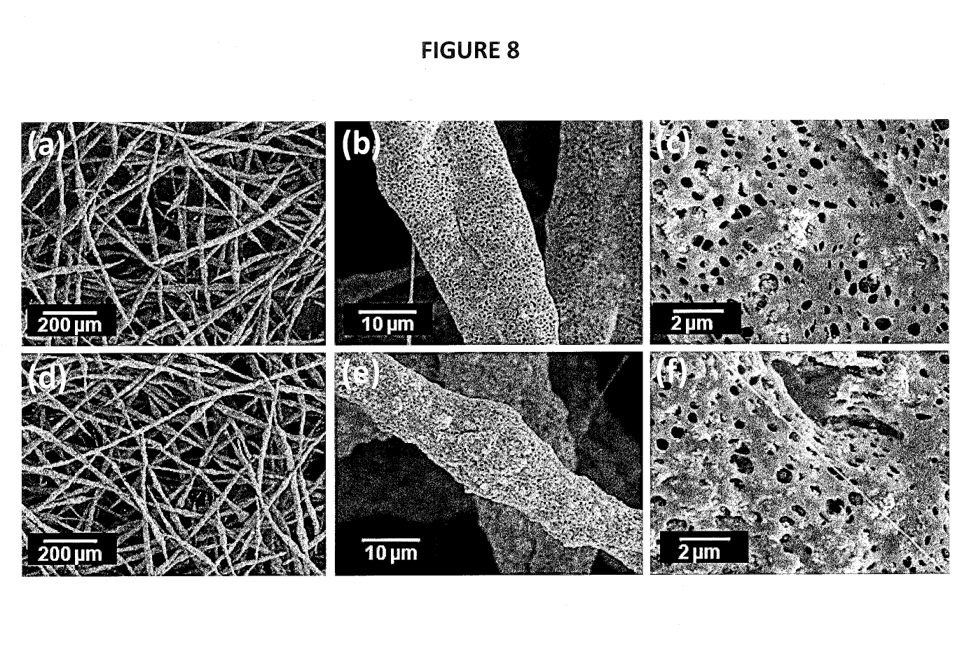

FIG. 7 shows representative SEM images of porous PLA fibers at varying magnification. The micrographs confirm that fibers have nano-scale size pores at their surface that are uniformly distributed throughout their length. SEM images of the porous PLA scaffolds incorporating 5 wt % and 10 wt % TCP are shown in FIG. 8, a-c and d-f, respectively. These SEM micrographs indicate that although addition of TCP changed the uniformity of fibers and the pores on their surface, pore formation was still able to occur during the electrospinning process.

Electrospun scaffolds, prepared as described above, were maintained under a fume hood for 24 hours to ensure the solvent was fully evaporated. The scaffolds were then peeled off the fiber collector (aluminum foil) and cut into 10×10 mm² squares. Thicknesses of scaffolds were measured with a Mitutoyo absolute micrometer, based on an average of at least 30 measurements. The weight of each sample was measured and maintained as close to equal to the mean average of all samples. Table 5 shows the properties of different samples.

TABLE 5
Physical Properties of Samples used for In Vitro Release Measurements

| | Average | Average | Average |
|---|---|---|---|
| | Diameter | Weight | Thickness |
| Scaffold Type | (nm) | (mg/cm²) | (μm) |
| Single Component | 450 ± 72 | 3 | 30 |
| Porous | 1020 ± 164 | 3 | 401 |
| Core-Sheath | 890 ± 125 | 3 | 50 |

Scaffolds then were put in 70% ethanol for 10 minutes. Scaffolds were rinsed three times with 1×phosphate buffered saline (PBS). Sterilized scaffolds were soaked in PBS in 12-well plates. Scaffolds were then removed from the surrounding media after one, two and four days; then removed from the PBS every 4 days up to 36 days. Experiments were performed in triplicate such that three different scaffolds were removed from the media at each time point. In total, this resulted in 33 samples for each structure for a combined total of 99 samples analyzed over the 36 day experimental duration. After removing from PBS, the scaffolds were then washed twice more with PBS to ensure no released calcium remained attached to the scaffolds. Experimental constructs were put in 0.5 mL of HCL and then frozen to the end of experiment. The PBS was removed from the plates and refrigerated. At the completion of the experiment all collected scaffolds were then removed from the freezer, placed in HCl, and kept on a rocker over night to ensure full scaffold digestion.

To quantify the amount of calcium, a microplate reader was utilized and absorbance measured at a wavelength of 550 nm to quantify calcium content. Scaffolds removed from HCL were also put under a fume hood for 3 days to ensure complete evaporation of solvent. Their weight was measured for determination of weight loss percentages of the scaffolds during the 36 day experimental duration. To obtain initial weight of scaffolds for weight loss calculations, three control scaffolds (single component, core-sheath, and porous) were also prepared and digested in HCL prior to immersion in PBS and degradation. The primary weight of pure PLA scaffolds (TCP content=0%) was quantified by calculating the mean weight of three samples. The weight loss percentage was calculated as follows:

$$Scaffold weight loss \% = \frac{\begin{matrix}Initial weight of scaffold - \\ Remaining weight of scaffold\end{matrix}}{Initial weight of scaffold} \times 100$$

The amount of TCP initially added to the scaffolds was determined by subtracting the primary weight of pure PLA from the weight of scaffolds with TCP (PLA with TCP scaffold weight=3 mg).

Core-sheath, porous, and single component fibers were quantified and found to equal 700 μg, 1200 μg and 900 μg, respectively. By including the molar mass of calcium ions (Ca²⁺) (40.08 g/mol), orthophosphates (PO₄³⁻) (94.97), and tricalcium phosphate (Ca₃(PO₄)₂) (310.18), we may calculate the weight of Ca²⁺ doped in the scaffolds. This calculation was performed and it was determined that core-sheath fibers had 271 μg, porous fibers 465 μg and single component fibers 349 μg. Since the weight of calcium remaining in the scaffolds was previously determined by the microplate reader, it was thus possible to determine the percentage of calcium ions released in the medium as indicated:

$$Release \% = \frac{\begin{matrix}Total weight {Ca}^{2 +} doped in scaffold - \\ Remaining weight {Ca}^{2 +} doped in scaffold\end{matrix}}{Total weight {Ca}^{2 +} doped in scaffold} \times 100$$

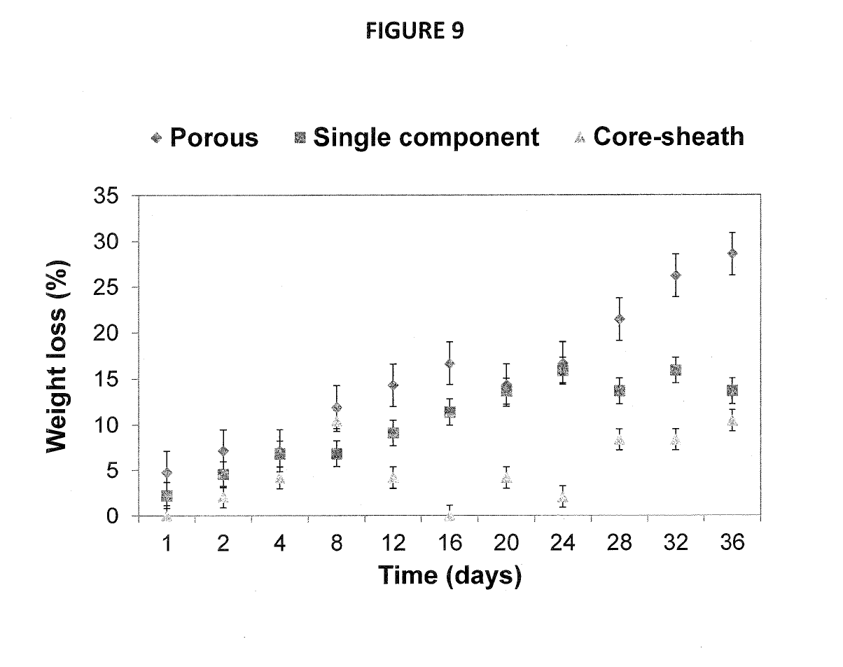

A straightforward means to determine the biodegradation of polymers is to determine their weight loss as a function of time. Calculating the percentage of weight loss for the three different fiber structures used in the TCP release analyses, it was found that the porous fibers exhibited the most weight loss during the 36 day experimental duration (about 30%) (FIG. 9). This is likely due to easier diffusion of medium through the fibers as a result of nanopores on the surface of the fibers.

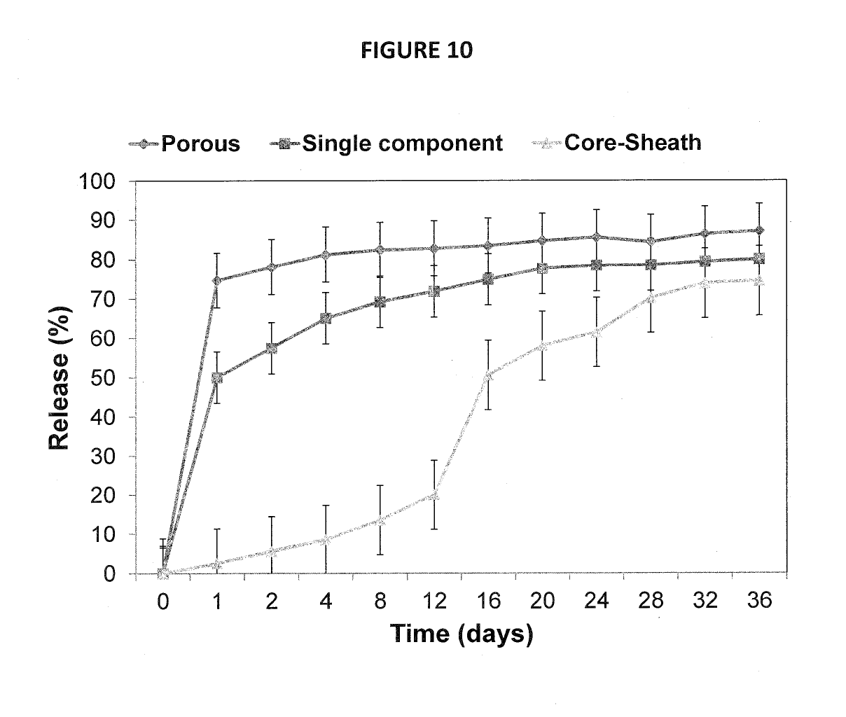

The minimum weight loss was associated with core-sheath fibers (less than 10%). Core-sheath nanofibers having larger fiber diameter in comparison with single component fibers exhibited less surface area, an important factor affecting the degradation rate of polymers (more surface area results in a faster degradation rate). Release profile indicated that the highest release occurred for porous fibers (FIG. 10). Although not intending to be limited by theory, it is believed that this high release is due to their weight loss (FIG. 9) and, thus, high release due to degradation of the polymer. The release profile for the single component fibers was similar to the porous but occurred at a slower rate. Core-sheath fibers, however, exhibited a distinctly different release profile. The initial release of core-sheath fibers was not as fast as porous and single component fibers. Again, it is believed that this slowed release is due to the sheath's inhibition of the release of particles from the core. Further, the rate of release for the core-sheath fibers remained fairly constant during 36 days of analysis. Therefore, core-sheath nanofibers show a great potential in drug delivery applications which require a steady release of drugs.

### Silver Nanoparticles (SNP)

Silver nanoparticles purchased from Nanocomposix are extensively purified and have an average diameter size of 20 nm They have mass concentration of 2.86 mg/mL and are stabilized in DI water. Silver nanoparticles were incorporated within PVA single-component fibers and within the core of core-sheath fibers comprising a PVA sheath and PEO core by incorporating silver nanoparticles in 0.2% or 0.6 weight percent within the electrospinning solution. The electrospinning parameters for single component and core-sheath fibers are indicated in Table 6.

TABLE 6
Electrospinning Parameters for Nanofibers with SNP

| Spinning Parameters | 0.2% SNP | 0.6% SNP |
|---|---|---|
| Voltage (kV) | 11-13 | 15 |
| Spinning distance (cm) | 15-16 | 13 |
| Feed rate (μm/min) | 0.6 | 0.4 |

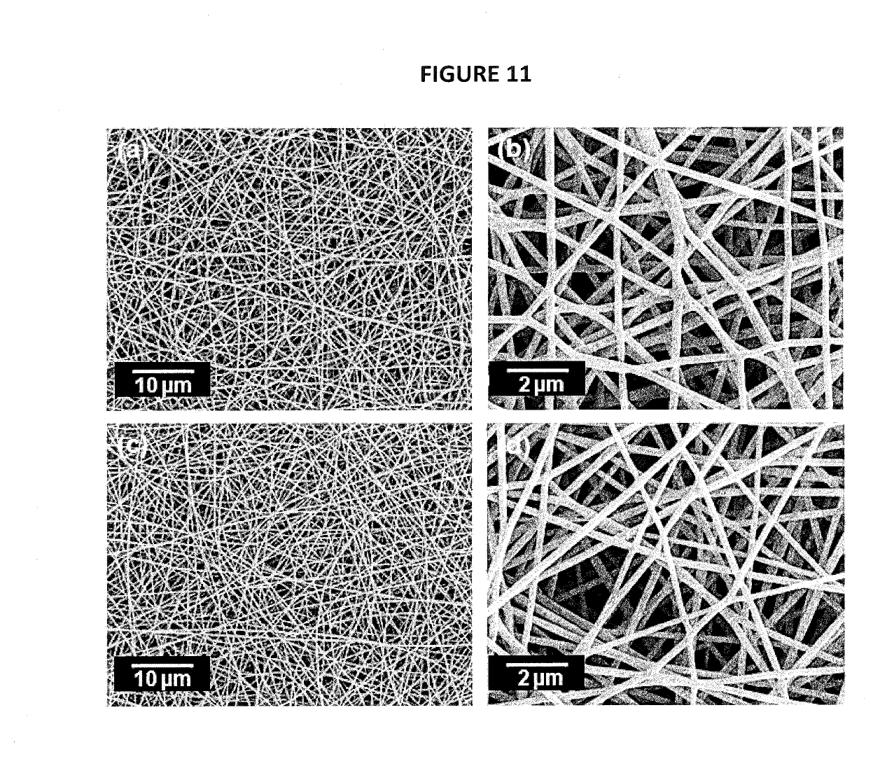

SEM micrographs of single component fibers loaded with 0.2 wt % and 0.6 wt % silver nanoparticles are shown in FIG. 11. FIGS. 11a) and 11b) show single component nanofiber materials with 0.2 wt % silver nanoparticles at magnifications of 2000× and 10,000× respectively. FIGS. 11c) and 11d) show single component nanofiber materials with 6 wt % silver nanoparticles at magnifications of 2000× and 10,000× respectively. These photos show that the resultant fibers have a uniform size distribution not just along their length but throughout the web. The mean fiber diameters for scaffolds made of 0.2 wt % and 0.6 wt % silver nanoparticles are 246.54 nm and 224.41 nm, respectively. This indicates that by increasing the silver nanoparticle percentage, the mean diameter of fibers reduces. This is potentially due to an increase in electrical conductivity of the electrospinning jet caused by addition of more silver nanoparticles since they have high electrical conductivity.

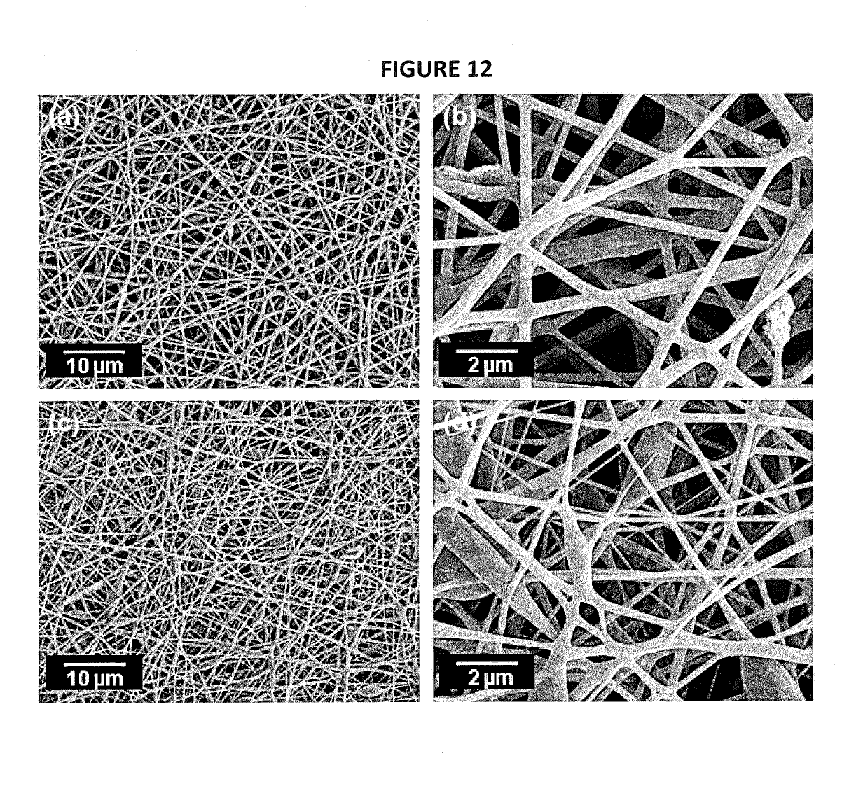

The PVA/PEO bicomponent fibers loaded with 0.2 wt % and 0.6 wt % of silver nanoparticles in the core component are shown in FIG. 12. FIGS. 12a) and 12b) show bicomponent nanofiber materials with 2 wt % silver nanoparticles at magnifications of 2000× and 10,000× respectively. FIGS. 12c) and 12d) show bicomponent nanofiber materials with 0.6 wt % silver nanoparticles at magnifications of 2000× and 10,000× respectively. The mean fiber diameter for mats comprised of 2 wt % and 6 wt % silver nanoparticles were 264.18 nm and 1323.9 nm, respectively. For core-sheath fibers, it was also shown that with a higher percentage of silver nanoparticles, the fiber diameter increased. Fiber diameter also increased with coaxial electrospinning Finally, increasing the percentage of silver nanoparticles led to formation of beads uniformly distributed throughout the web (see FIGS. 12c and 12d). Via increasing SEM magnification, more detailed observation of silver nanoparticles within the core-sheath fibers was possible. FIG. 4 provides an SEM micrograph of a bead in a core-sheath scaffold, with a loading of 0.6 wt % silver nanoparticles, showing these are fully encapsulated with silver nanoparticles. The silver nanoparticles were incorporated within the core fiber component and are seen in this Figure to be contained within a “bead” formed during formation of the composite fiber. This Figure illustrates that fibers having portions filled with silver nanoparticles (i.e., having silver nanoparticles on the interior of the fibers, e.g., encapsulated by the fibers) can be prepared by the methods described herein. Such structures are beneficial as they can provide for more extended release of the silver nanoparticles as compared with structures having silver nanoparticles on the exterior of the fibers.

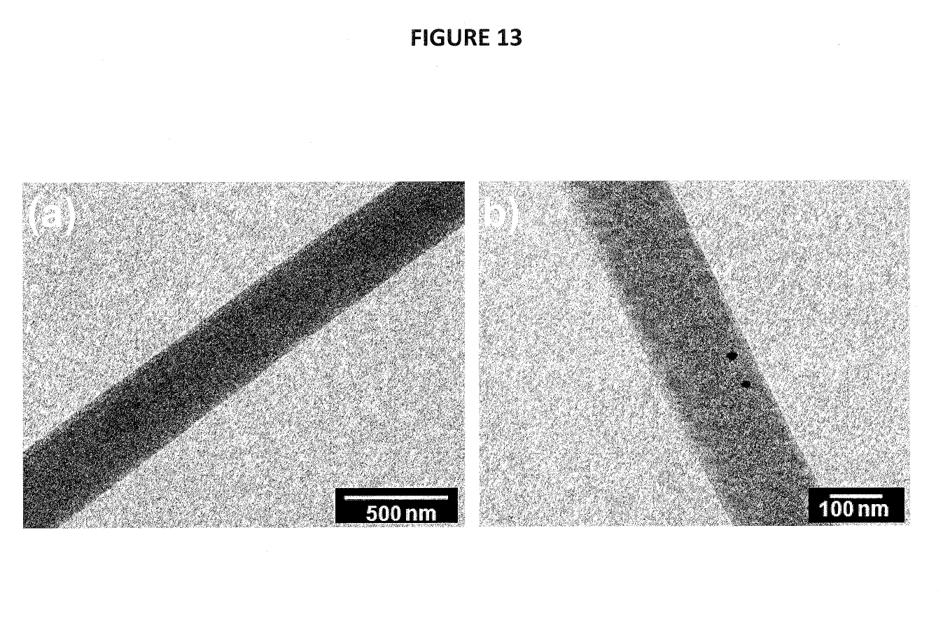

In order to observe the two different polymers (PVA and PEO) in a core-sheath structure, TEM micrographs were obtained: FIG. 13 shows two of these photos for fibers loaded with 2 wt % silver nanoparticles. As can be seen in these photos, the sheath layer is very thin. This is possibly a result of the low feed rate utilized in this experiment. Variations in these thicknesses will be acquired by changing the feed rates and/or varying molecular weights of the polymers.

### Ibuprofen (IB)

Ibuprofen is a nonsteroidal anti-inflammatory drug (NSAID) used to relieve pain and reduce excessive inflammation of the wound bed which can prevent healing. Anti-inflammatory activity of ibuprofen appears to be achieved mainly through inhibition of the enzyme cyclooxygenase (COX) and provides relief from the symptoms of inflammation and pain.

Ibuprofen in the form of ibuprofen sodium salt was purchased from Sigma Aldrich and was dissolved with PLA in chloroform and dimethylformamide (DMF) with a ratio of 1:3 (DMF mL/chloroform mL) The solution was mixed for 24 hrs at 80° F. The solution was then spun in a single component electrospinning setup with electrospinning parameters as indicated in Table 7.

TABLE 7
Electrospinning Parameters for Nanofibers with Ibuprofen

| Spinning Parameters | | | | |
|---|---|---|---|---|
| PLA wt %, IB wt % | 15, 3 | 15, 6 | 12, 4 | 12, 8 |
| Voltage (kV) | 14 | 14 | 14 | 14 |
| Spinning distance (cm) | 15-13 | 13-15 | 13-15 | 13-15 |
| Feed rate (μm/min) | 0.8 | 0.6 | 0.3 | 0.1 |

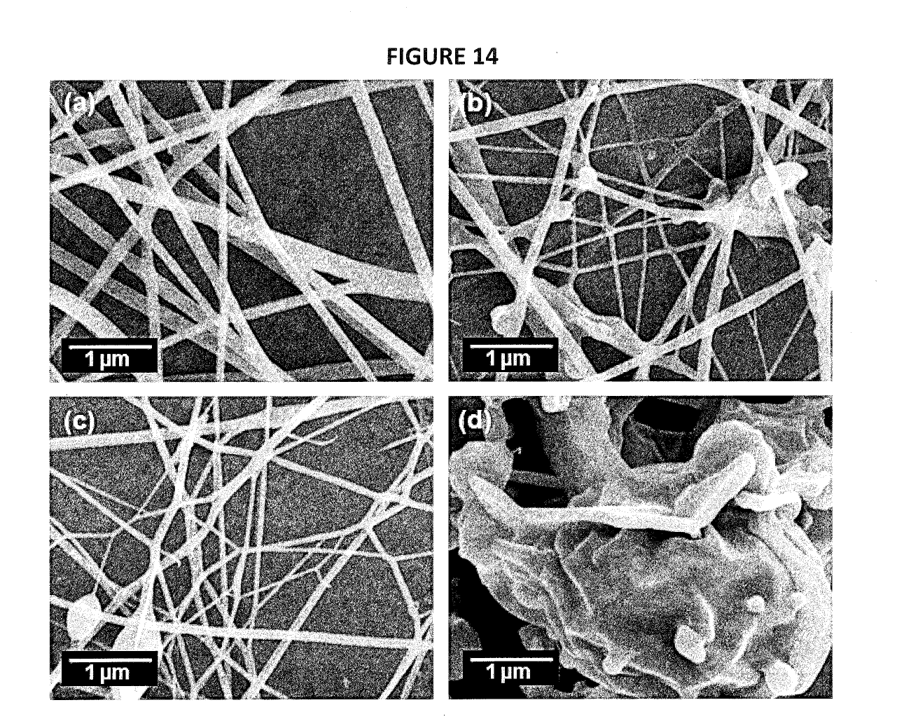

Nanofibers formed successfully from the PLA/ibuprofen solutions, with the exception of the solution comprising 12% PLA/8% ibuprofen. SEM micrographs were obtained to evaluate fiber formation for each sample. In FIG. 14, micrograph (A) shows nanofibers prepared from a solution of 15 wt % PLA, 3 wt % ibuprofen; micrograph (B) shows nanofibers prepared from a solution of 15 wt % PLA, 6 wt % ibuprofen; micrograph (C) shows nanofibers prepared from a solution of 12 wt % PLA, 4 wt % ibuprofen; and micrograph (D) shows nanofibers prepared from a solution of 12 wt % PLA, 8 wt % ibuprofen; As shown in FIG. 14, increasing the percent of ibuprofen led to bead formation.

Table 8 provides the mean values and standard deviations of fiber diameter for different compositions of PLA-ibuprofen fibers as calculated from at least 60 measurements per calculation of each average. The standard deviation depicts high variance in the samples suggesting multiple factors, such as temperature and humidity affecting the fiber formation. A general trend that was seen in samples comprised of 15% PLA solution was a decrease in fiber diameter with increasing percentage of ibuprofen. It was also seen that 15% PLA by weight produced larger fiber diameters in comparison to the 12 wt % PLA solution.

TABLE 8
Fiber Diameter Measurements for PLA Scaffolds Loaded with Ibuprofen

| | | | |
|---|---|---|---|
| PLA wt %, IB wt % | 15, 3 | 15, 6 | 12, 4 |
| Average diameter | 143.27 | 91.65 | 63.62 |
| Standard Deviation | 49.89 | 54.31 | 31.63 |

In order to confirm the presence of ibuprofen in the sample as well as ensure the chemical integrity of Ibuprofen after being exposed to a high voltage source, nuclear magnetic resonance (NMR) spectroscopy for two different samples (15 wt % PLA, 3 wt % ibuprofen and 12 wt % PLA, 4 wt %) was performed. NMR spectroscopy confirmed that Ibuprofen was present in the nanofiber scaffolds and that its chemical integrity remained intact.

### Example 3

### Comparison of Silver-Containing Scaffolds

All fiber morphologies were created using FDA approved, biocompatible, biodegradable polylactic acid (PLA) (70,000 g/mol) using an electrospinning system. Two different antimicrobial agents, Silvadur ET and silver nanoparticles (average diameter of 20 nm), as described in the previous examples were incorporated in the polymeric fibers. The antimicrobial activity of the scaffolds against different bacteria was determined by two different methods: (i) qualitative evaluation using an agar diffusion assay (parallel streak method) and (ii) quantitative evaluation in a liquid medium.

Qualitative analysis of the antimicrobial activity of the scaffolds treated with Silvadur ET and silver nanoparticles was evaluated using the parallel streak method (Antibacterial Activity Assessment of Textile Materials—AATCC 147). Scaffolds were placed on a substrate of bacteria in an agar plate and incubated overnight at 37° C. Inoculants used for evaluation were both Gram positive (Staphylococcus aureus) and Gram negative (Escherichia coli J53) bacteria along with a silver resistant bacteria (Escherichia coli J53pMG101). The bactericidal activity was visually assessed to observe a clear zone of inhibition around the fiber mat after an overnight incubation.

Quantitative analysis of the antimicrobial activity of the scaffolds was evaluated by cutting the scaffolds into 1 cm² squares, sterilizing with 70% ethanol, and soaking in Mueller-Hinton (MH) broth inoculated with Escherichia coli bacteria with initial concentration of 10⁶ CFU/mL The suspensions were incubated at 37° C. for one week. Bacterial concentration after incubation was determined by spreading on tissue culture plates. Non-treated PLA scaffolds were included as negative controls.

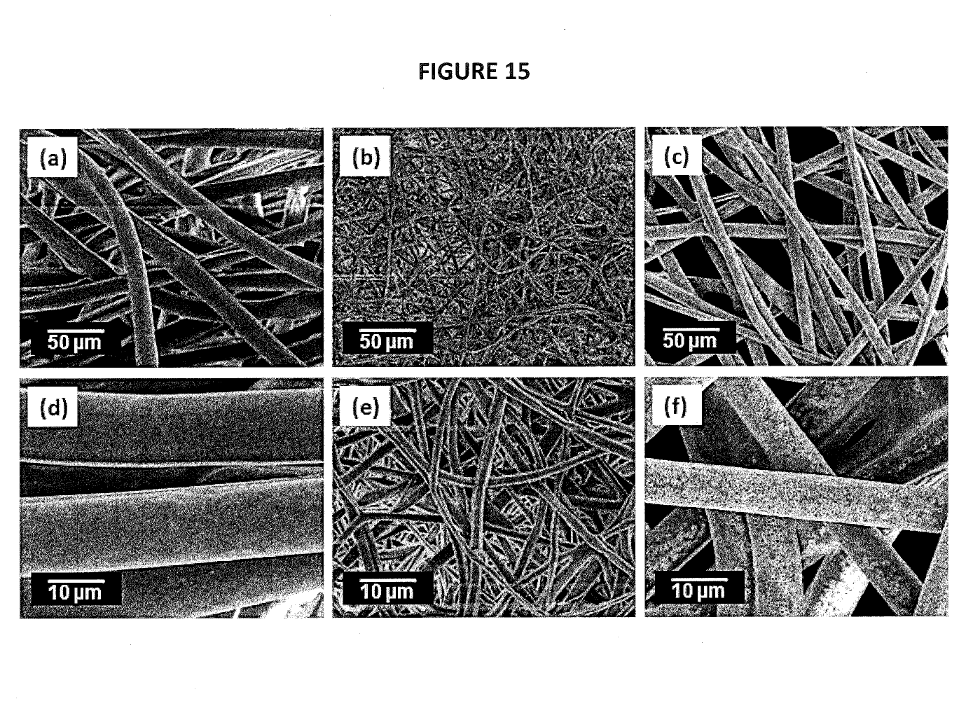

Three different fiber morphologies: spunbond fibers, electrospun single component nanofibers and electrospun porous fibers were coated with Silvadur ET solution. Scanning electron microscopy analysis of these fibers indicated a uniform distribution of coated polymer on the fibers. An exciting result was that the polymer coating did not cover the pores on the surfaces of the porous fibers and the majority of the pores were visible via SEM. This was a desirable, but not necessarily anticipated, result that confirmed coating with Silvadur ET did not negatively impact the desired porous fiber morphology for use of these fibers in antimicrobial/antibacterial applications. FIGS. 15a and 15d show spunbond fibers, FIGS. 15b and 15e show electrospun single component nanofibers, and FIGS. 15c and 15f show electrospun porous fibers coated with Silvadur ET.

Some physical properties of the porous fibers were compared with the regular nanofibers, which have solid surfaces. Regular PLA nanofibers exhibited a fiber diameter of 450±72 nm, a multipoint BET of 2.181 m²/g, and a moisture pickup of 172%. Porous PLA nanofibers exhibited a fiber diameter of 1020±164, a multipoint BET of 1.541 m²/g, and a moisture pickup of 372%. In particular, it was found that porous fibers have less surface area, which could be due to their larger fiber diameter. However, the moisture pick-up for porous fibers is significantly greater than regular nanofibers. This could be related to better diffusion of moisture through the nano-sized pores dispersed throughout the surfaces of these fibers. Therefore, it is anticipated that porous fibers will uptake significantly greater amounts of antimicrobial compounds via enhanced diffusion through the scaffold and can therefore deliver significantly greater amounts of antimicrobial compounds at a wound site. Simultaneously, they have enhanced wicking capabilities for high absorbency to promote a drier wound site. With a combination of sustained release that can be achieved with the use of a solid core and high initial release of antimicrobial through the use of a porous sheath, both immediate and sustained antimicrobial/antibacterial activity should be achieved while simultaneously promoting absorption.

Antimicrobial activity results indicated that scaffolds treated with Silvadur ET solution inhibited the growth of both gram negative (Escherichia coli) and gram positive bacteria (Staphylococcus aureus). However, as desired, these scaffolds had almost no effect on inhibiting the growth of silver resistant bacteria (E. Coli J53pMG101). These results confirmed that the antimicrobial properties of scaffolds treated with the Silvadur ET solution were due to the release of silver ions. Therefore, the additional component in this solution that was killing the bacteria in solution is removed when the treated scaffolds are dried.

Measuring the zone of inhibition (the diameter of the circle in which no visual bacterial growth can be seen), it was found that porous fibers exhibited a larger zone of inhibition than regular fibers. In the presence of staphylococcus aureus, PLA nanofibers exhibited a zone of inhibition of 3.5-4 mm, whereas porous PLA fibers exhibited a zone of inhibition of 4.5-5 mm. In the presence of Escerichia coli J53, PLA nanofibers exhibited a zone of inhibition of 3.5-4 mm, whereas porous PLA fibers exhibited a zone of inhibition of 4.5-5 mm. This could be as a result of the porous fiber scaffold absorbing more Silvadur ET solution, a desirable outcome based on physical differences between the fiber types as noted previously.

Scaffolds coated with Silvadur ET along with control PLA scaffolds were kept in Escherichia coli bacteria solution for a week. After one week, the concentration of bacteria in the solutions with and without scaffolds (coated or uncoated) was calculated and the percentage of bacteria reduction (Reduction %) was calculated using following formula:

Reduction %=100(B−A)/B,

where A is the number of bacteria recovered from the inoculated coated PLA scaffold in the tube after one week, and B is the number of bacteria recovered from the inoculated uncoated control PLA scaffold. The Reduction % calculated for PLA scaffolds coated with Silvadur ET solution was 100%. A visual comparison was done of titre density that relates to the differing concentration of bacteria in the two glass tubes having coated and uncoated PLA scaffolds compared to a solution with no scaffold.

To further confirm release properties of the scaffolds, they were doped with silver nanoparticles instead of Silvadur ET to evaluate their antimicrobial activities against the three bacteria analyzed with Silvadur ET treated scaffolds. For all antimicrobial analyses, no definitive zone of inhibition was present; however, for E coli treated agar, a very fine zone of inhibition was noted upon visual inspection. These results confirm that there was minimal silver on the surface of these scaffolds. Transmission electron micrograph (TEM) analysis of these fibers further indicated that the silver nanoparticles were well encapsulated inside the fibers, as would be desired for a sustained, controlled release over time in response to degradation of the polymer in a liquid medium as opposed to a burst release.

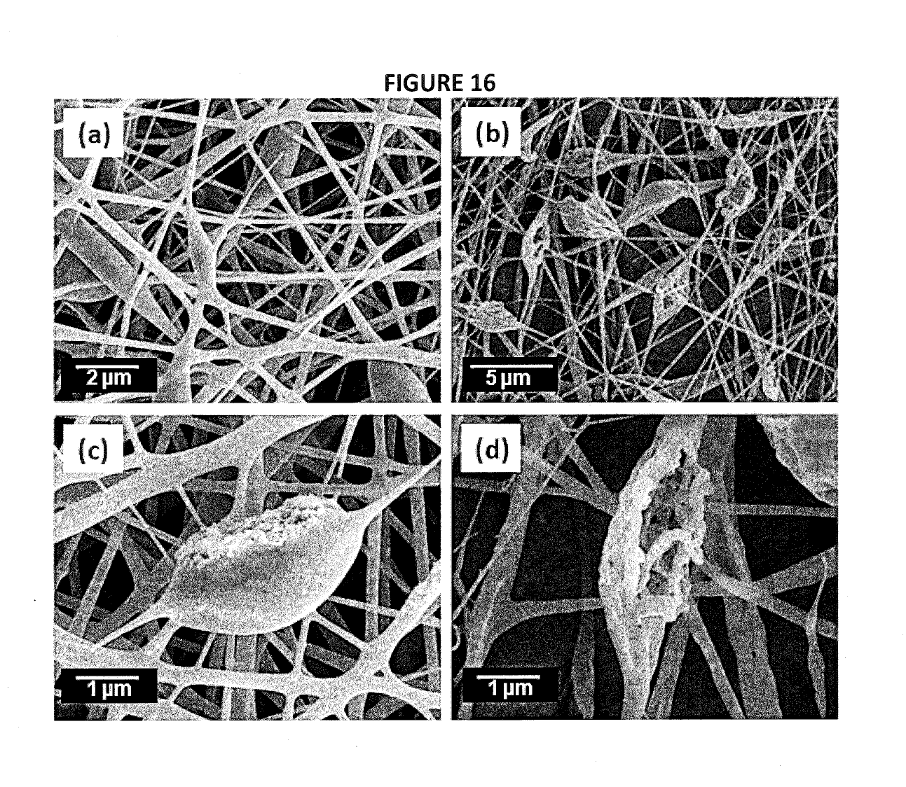

The quantitative analysis of PLA fibers containing silver nanoparticles indicated that maintenance of these fibers in a liquid medium over time will allow the encapsulated nanoparticles to be released from the fibers and inhibit bacterial growth. The Reduction % after one week for these scaffolds was calculated and found to be 84%. FIG. 16 shows the surface morphology of these scaffolds before (16a) and 16c)) and after (16b) and 16d)) the release of nanoparticles.

### Example 4

### Comparison of Scaffolds Having Human Adopise Derived Stem Cells Seeded Thereon

In this study, PLA (MW: 70,000 g/mol) having varied concentrations was dissolved in different solvent systems to prepare polymeric solutions. In each case the accurate weight percentage of polymer was weighed and dissolved in the appropriate solvent. The mixture was stirred on a magnetic stirrer plate for at least 12 hours at 80° C. until a homogeneous solution was obtained. Polymer solutions were used within 24 hours of preparation to eliminate evaporative loss of solvent and consequent change in solution concentration. Single component nanofibers, core-sheath fibers, and porous fibers were prepared. BET (Brunauer, Emmett, Teller) analysis was performed to quantify the surface areas of each of the fiber types. Samples were degassed for 5 hours before each BET test.

Human adipose derived stem cells (hASC) were obtained from excess human adipose tissue obtained from liposuction procedures on a 36 year old Caucasian female in accordance with an approved IRB protocol (IRB-04-1622) at University of North Carolina, Chapel Hill. Human ASC were isolated as previously reported in Gao et al., Textile Res. J. 2008, vol. 78 and Zuk et al., Tissue Engineering, 2001, Vol. 7, which are incorporated herein by reference. Human ASC were pre-cultured in complete growth medium (CGM) to 80% confluency in 75 cm² tissue culture flasks, trypsinized, suspended in CGM, and seeded on the porous and regular single component scaffolds at an initial cell seeding density of 2×10⁴ cells/cm². Culture medium was changed every 3 days for cell seeded scaffolds. Cell viability was determined on days 1 and 14 using a fluorescent method (Live-Dead Assay Cytotoxicity Kit for mammalian Cells; Molecular Probes, Eugene, Oreg.). Specifically, cell seeded scaffolds were rinsed twice with 1×PBS and 4 mM calceinacetoxymethyl ester (AM) and 4 mM ethidiumhomodimer were added to the cytoplasm of live cells green and the nuclei of dead cells red, respectively. The samples were then incubated for 20 minutes while protected from light.

Stem cell proliferation was determined with a cell viability assay (alamarBlue, AbDSerotec, Raleigh, N.C.) at days 1, 3, 7, and 14 post-seeding. AlamarBlue, at a volume of 10% of the culture medium, was added to each well 5 hours before each measurement. After incubation of the alamarBlue, 200 μL of each sample was taken in triplicate and the absorbency read at 600 nm using a microplate reader (TecanGENios, Tecan, Switzerland) (a greater alamarBlue reduction indicates greater cell proliferation).

To quantify the hASC-mediated calcium accretion in the scaffolds, scaffolds were washed twice with 1×PBS then soaked in 0.5 N HCl and the supernatant tested using the Calcium Liquicolor Assay (Stanbio, Boerne, Tex.). In order to normalize the cell mediated calcium accretion per cell, the protein content in cells was quantified using BCA protein assay for normalization purposes. In order to visualize cell mediated calcium accretions, scaffolds were washed with 1×PBS, then fixed with 4% formalin for 20 min, and stained with 40 mM Alizarin Red S, for 3 min, and rinsed with deionized water five times to remove any unbound stain. Images were captured with a Leica (Wetzlar, Germany) EZ 4D Digital Dissecting Scope.

The silver nanoparticles had a spherical shape with mean diameter of 20 nm (Nanocomposix, USA) and atomic molarity of 9.25 mM. The mass concentration of these nanoparticles dispersed in water was 1.0 mg/mL. The Silvadur ET solution containing 2.6-3.1 wt % silver ions was used at its original concentration (1.0 mg/mL) Silver nitrate was also dissolved in distilled water to make 1 mg/mL solution.

A gram negative (Escherichia coli J53) and gram positive (staphylococcusaureus, a common bacteria that causes skin infections) bacteria were employed for the experiment. Cation-adjusted Mueller-Hinton (MH) broth and agar (Difco Laboratories, Detroit, Mich., USA) were used to prepare bacterial cultivating medium. Isolated bacterial colonies were grown overnight in an incubator (37° C., 5% CO₂) from frozen samples on an agar plate. For Ag-resistant bacteria (E. coli J53 [pMG101]) 100 mg/ml ampicillin sodium salt (Fisher Scientific, USA) was also added to the agar plates. The bacterial colony was suspended in phosphate buffered saline (PBS) to get 0.5 McFarland (10⁵ CFU/ml). Microplates were incubated at 37° C. and shaken at 200 rpm for 24 h. To control the accuracy of bacterial seeding density, bacteria were diluted in PBS at 10³, 10⁴, 10⁵, and 10⁶ and plated overnight.

The minimum inhibitory concentration (MIC) test was conducted to measure the efficiency of silver based antimicrobials against each bacterial isolate. A sterile round-bottom plastic 96-well plate containing 100 ml of serially 1:2 diluted concentrations of antimicrobial solutions was inoculated with 100 ml of 5-8×10⁵ CFU/ml of each bacterial isolate. Each antimicrobial sample was tested at 15 serially diluted concentrations starting at their original highest concentration (0.015-1000 μg/ml). After the microplates were incubated for 24 h in incubator, the MIC was recorded to be the lowest concentration of antimicrobial which shows no visible growth. After 24 h of incubation, 10 μl of the suspension from all of the clear wells (showing no bacterial growth) was dropped onto a MH agar plate and incubated for 24 h. The minimum bactericidal concentration (MBC) was determined by the concentration that failed to kill bacteria.

Using the parallel streak method (Antibacterial Activity Assessment of Textile Materials—AATCC 147) the antimicrobial properties of scaffolds treated with Silvadur ET and silver nanoparticles were tested. Scaffolds were placed on a lawn of bacteria in an agar plate and incubated overnight at 37° C. The inoculants used were both Gram positive (Staphylococcus aureus) and Gram negative (Escherichia coli J53) plus the silver resistant bacteria (Escherichia coli J53pMG101). The bactericidal activity showed a clear zone of inhibition around the fiber mat after an overnight incubation.

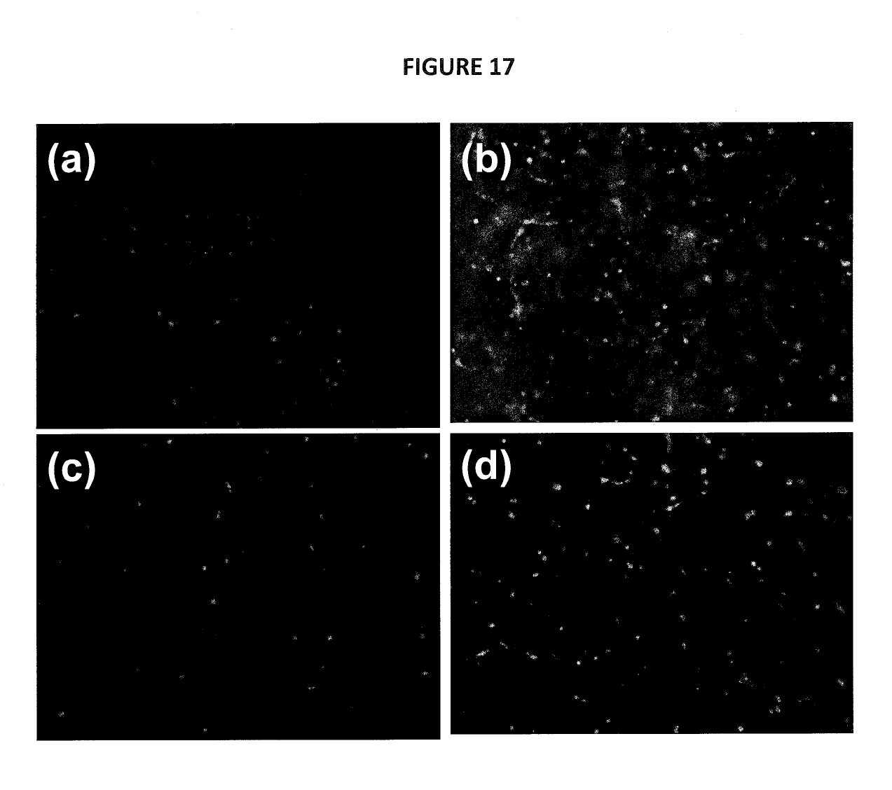

Typically, surface area of the fibers directly depends on their fiber diameter and surface morphology. Thinner fibers have a higher surface area. Also, surface porosity of fibers increases their surface area significantly. The BET results indicated a higher surface area for regular nanofibers compared to porous fibers (regular PLA fibers with a fiber diameter of 450±72 2 nm exhibited a multipoint BET of 2.181 m²/g, whereas porous PLA fibers with a fiber diameter of 1020±164 1 exhibited a multipoint BET of 1.541 m²/g. This observation emphasizes the stronger effect of fiber diameter in determining the surface area of fibers as compared to surface porosity. The viability analysis of stem cells grown on the scaffolds on day 1 and 14 exhibited higher number of cells at 14 compared to day 1 for both regular and porous fibers. Furthermore, cell viability for regular fibers was higher than porous fibers after 14 days. FIG. 17 provides viability images of human adipose drive stem cells seeded on regular (a, b) and porous (c, d) fibers on day 1 (a, c) and on day 14 (b, d). Bright spots show live cells.

AlamarBlue reduction was used to determine cell proliferation on porous and regular nanofibers in complete growth medium (CGM) and osteogenic differentiation medium (ODM). The results showed that cell proliferation was higher for regular fibers than porous fibers regardless of whether cells were cultured in CGM or ODM. Alizarin red staining was used to visualize cell mediated calcium accretion on the scaffolds on days 14 and 21. After 21 days, osteogenic differentiation clearly was seen in all scaffolds. However, the highest calcium content was on scaffolds created with regular nanofibers. Calcium quantification results confirmed alizarin red staining results. Moreover, these results indicated higher calcium content per each cell for porous fibers than spunbond scaffolds.

As shown in Tables 9 and 10, minimum inhibition concentrations and minimum bactericidal concentrations were calculated for the silver nanoparticles, Silvadur ET solution, and AgNO₃ on each bacterial strain (n=6). The results showed that the Silvadur ET solution is significantly more efficient in inhibiting bacterial growth in comparison with silver nanoparticles. Moreover, more antimicrobials of any kind are required to inhibit the growth of the same number of S. aureus bacteria as compared to amount required to inhibit growth of E. coli bacteria. Plasmid pMG101 is a silver resistant plasmid containing nine genes that also confers resistance to mercury, tellurite and several antibiotics. As bacterial contact with Ag increases, the number of Ag-resistant bacteria will correspondingly increase. Therefore, silver resistant bacteria have been reported in Ag-saturated environments. Observation of silver resistant E. coli J53 (pMG101) in the presence of silver nanoparticles and silver nitrate confirmed its resistance to silver. While inoculation of Silvadur ET solution with this bacterium suggests that there are additional components other than silver ions in this solution that are responsible for killing this strain of silver-resistant bacteria.

TABLE 9
Values of Minimum Inhibitory Concentrations (MIC)

| | Staphylococcus | | |
|---|---|---|---|
| | aureus | Escherichia | E. Coli |
| Silver Type | (μg/ml) | coli J53 | J53pMG101 |
| Silver nanoparticles | 125 | 62.5 | — |
| Silvadur ET | 0.24 | 0.12 | 0.98 |
| Silver nitrate | 15.62 | 7.81 | — |

TABLE 10
Values of Minimum Bacterial Concentrations (MBC)

| | Staphylococcus | | |
|---|---|---|---|
| | aureus | Escherichia | E. Coli |
| Silver Type | (μg/ml) | coli J53 | J53pMG101 |
| Silver nanoparticles | 125 | 62.5 | — |
| Silvadur ET | 0.98 | 0.24 | 3.91 |
| Silver nitrate | 31.25 | 15.62 | — |

Test results indicated that scaffolds treated with Silvadur ET solution inhibited the growth of both gram negative (Escherichia coli) and gram positive bacteria (Staphylococcus aureus). However, these scaffolds had almost no effect on inhibiting the growth of silver resistant bacteria (E. Coli J53pMG101). These results showed that the antimicrobial properties of scaffolds treated with Silvadur ET solution was due to the release of silver ions. Therefore, the additional component in this solution that was killing the bacteria in solution can be removed when the treated scaffold is dried. Again, as in Example 3, above, the zone of inhibition was higher for both staphylococcus aureus and Escherica coli J53 in the presence of porous PLA nanofibers than traditional PLA nanofibers. In the presence of staphylococcus aureus, PLA nanofibers exhibited a zone of inhibition of 3.5-4 mm, whereas porous PLA fibers exhibited a zone of inhibition of 4.5-5 mm. In the presence of Escherica coli J53, PLA nanofibers exhibited a zone of inhibition of 3.5-4 mm, whereas porous PLA fibers exhibited a zone of inhibition of 4.5-5 mm.

### Example 5

### Study of Ibuprofen Release

The ibuprofen release of various scaffolds was studied. The acid form of ibuprofen was loaded into 10% PLA solution in DCM/DMF (3:1) to make nonporous PLA fibers including 10% and 20 wt % ibuprofen. The confirmation of the chemical integrity of Ibuprofen after being exposed to a high voltage source was analyzed via ¹H NMR. SEM photos were taken to evaluate the fiber morphology. The fiber mats were cut into 1 cm² squares and washed with 70% ethanol and then submerged in pure water to release the ibuprofen. The release was taken over 264 hours and evaluated with UV-vis spectroscopy at wavelength of 280 nm.

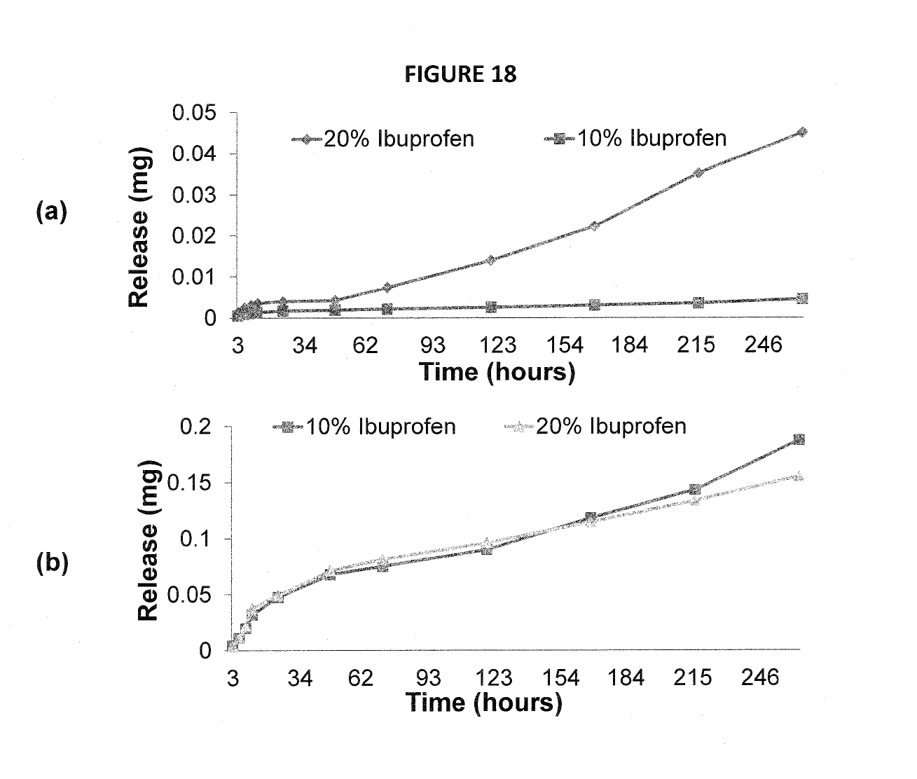

Results indicated that for scaffolds loaded with either 10% or 20% ibuprofen, more ibuprofen was released at 37° C. than at room temperature. Furthermore, scaffolds loaded with 20% Ibuprofen exhibited a faster rate of release compared to scaffolds containing 10% ibuprofen in room temperature. At body temperature (37° C.), the scaffolds loaded with 10% and 20% ibuprofen exhibited almost the same rate of release. FIG. 18 details the findings of the ibuprofen release profiles at room temperature (FIG. 18a) and 37° C. (FIG. 18b).

### Example 6

### Studies of Scaffolds Incorporating Silvadur ET

All fibrous scaffolds were produced from a polylactic acid (PLA) (70,000 g/mol) solution in chloroform and dimethylformamide (3:1) using a custom electrospinning system. In order to coat the scaffolds with the antimicrobial agent, scaffolds were maintained in different concentrations of Silvadur ET for one hour. They were then removed and dried under a fume hood for 24 hours. The antimicrobial activity of the scaffolds against different bacteria was determined via two approaches: (i) qualitative evaluation using an agar diffusion assay (parallel streak method); and (ii) quantitative evaluation in a liquid medium. Cell studies were performed using human dermal fibroblasts derived from adult skin (2^{nd} passage) purchased from Lonza (USA). PLA nanofibers coated with Silvadur ET, containing different concentrations of silver (250, 125, 62.5 and 31.25 μg/ml), were cut into circles (d=1.6 cm). Nanofiber samples were then sterilized with ethylene oxide for 12 hr and soaked in fibroblast growth medium (FGM) for 24 hr. Cells were then seeded on the scaffolds at an initial density of 4×10⁴ cells per scaffold and incubated for 7 days in fibroblast growth medium (FGM) containing 2% serum. Cell culture medium changes were performed every three days. Cell viability was determined on days 1, 4 and 7 using a fluorescent method (Live-Dead Assay Cytotoxicity Kit for mammalian cells; Molecular Probes, Eugene, Oreg.). Specifically, cell seeded scaffolds were put in FGM with the addition of 4 mM calceinacetoxymethyl ester-AM (staining the cytoplasm of live cells green) and 4 mM ethidiumhomodimer (staining the nuclei of dead cells red).

The samples were then incubated for 20 minutes while protected from light. Fibroblast cell proliferation was determined with a cell viability assay (AlamarBlue, AbDSerotec, Raleigh, N.C.) at days 1, 4 and 7 post seeding of cells on scaffolds. AlamarBlue, at a volume of 10% of the culture medium, was added to each well 7 hours before each measurement. After incubation with AlamarBlue, 200 μL of each sample was taken in triplicate and the absorbency read at 600 nm using a microplate reader (TecanGENios, Tecan, Switzerland). A greater AlamarBlue reduction % indicated greater cell proliferation. SEM was used to study the morphology of fibroblast cells on the scaffolds. After 7 days cell seeding, samples were fixed in 10% buffered formalin for 30 min and then dehydrated with a graded concentration (50-100% v/v) of ethanol. Dehydrated scaffolds were immersed in hexamethyldisilazane for 15 min and let dry overnight under a fume hood. After drying the samples, they were coated with gold using sputter coating to observe the morphology of cells using SEM. Quantitative antimicrobial analyses were performed for 24 hr on scaffolds coated with different concentrations of Silvadur ET. The results showed 100% reduction for all the scaffolds in the presence of two different bacteria, E. Coli and Staphylococcus. Qualitative analysis also indicated that there was no significant difference between the zone of inhibition formed around scaffolds coated with different concentrations of Silvadur ET.

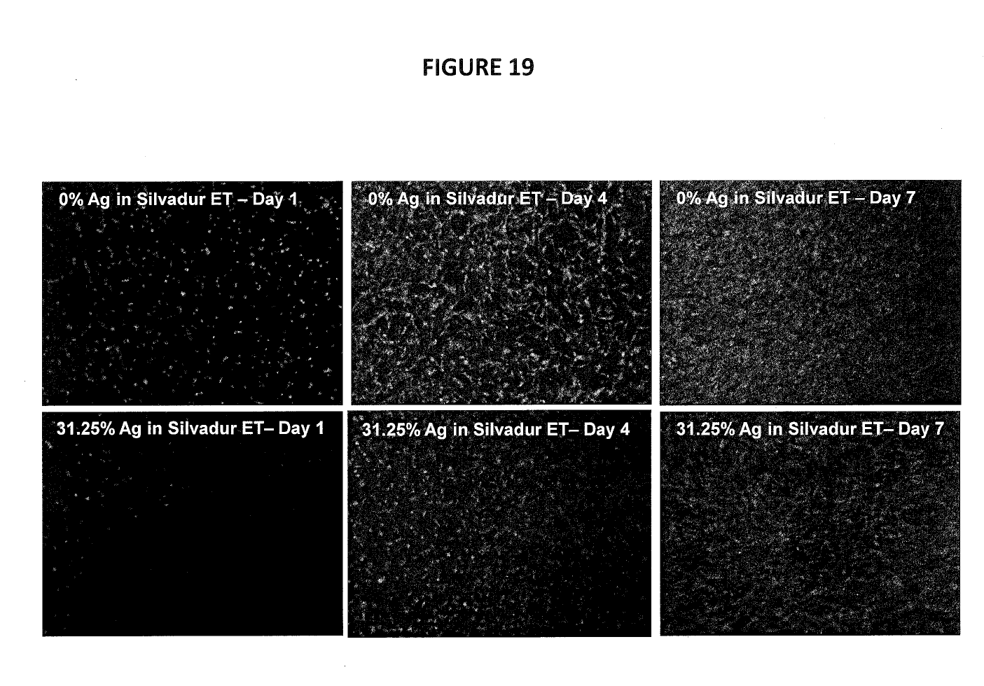

Viability analyses of human dermal fibroblast cells grown on the treated PLA scaffolds on days 1, 4 and 7 indicated that the cells were not viable on the scaffolds coated with Silvadur ET at a concentration of 250 μg/ml containing 125 μg/ml silver. A few viable cells were seen on the scaffolds coated with a more dilute Silvadur ET solution (silver concentration: 62.5 μg/ml). However, the majority of cells were viable on nanofibers coated with the more dilute Silvadur ET concentration containing 31.25 μg/ml silver. FIG. 19 shows fluorescent images of viable cells on scaffolds coated with Silvadur ET containing 31.25 μg/ml silver as compared to the uncoated PLA control scaffolds. These images demonstrate that the cells adhered to both coated and non-coated scaffolds and, in both instances, better adhesion of cells was achieved with extended culture duration. Keratinocyte cells were seeded only on scaffolds on which the fibroblasts were viable (silver concentration in coating solution: 62.5, 32.25 μg/mL) plus one other scaffold coated with lower concentration of silver in Silvadur ET solution. The majority of cells were viable on all three scaffolds.

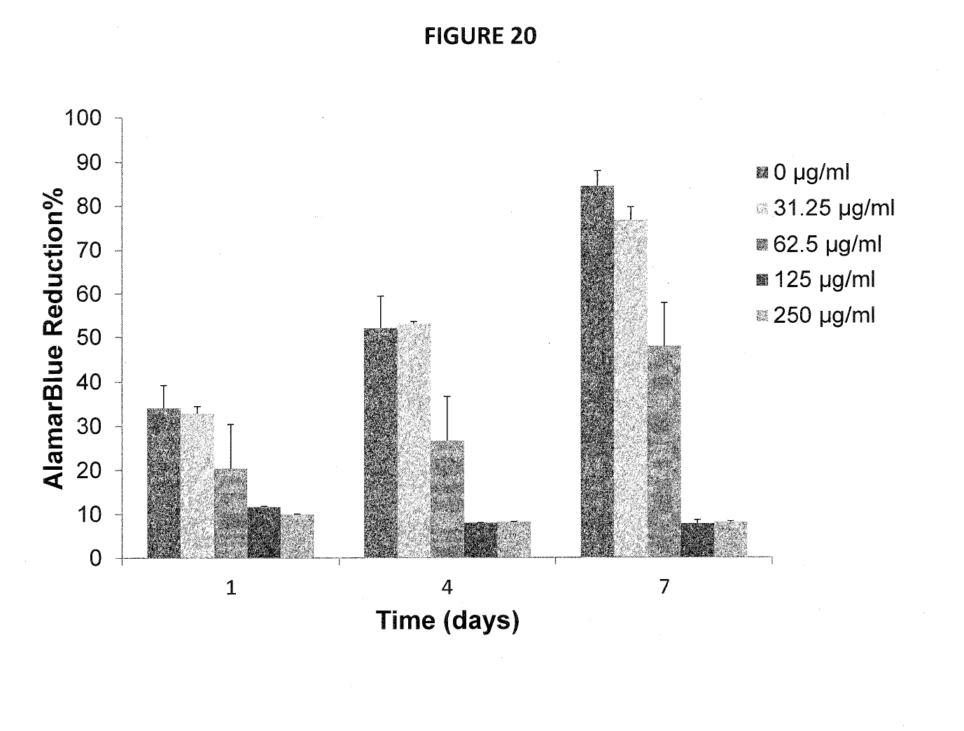

AlamarBlue results indicated that cell proliferation was diminished on PLA scaffolds coated with higher concentrations of Silvadur ET. The reduction %, indicative of cell proliferation, was almost constant for scaffolds coated with the most concentrated Silvadur ET solution (250 and 125 μg/ml silver), indicating no cell proliferation on these scaffolds. These results support the viability analyses, which indicated no viable cells on these scaffolds. However, there was no significant difference between cell proliferation on scaffolds coated with the less concentrated Silvadur ET solution (31.25 μg/ml) and the uncoated pure PLA scaffold controls at any time point (FIG. 20). FIG. 20 shows the AlamarBlue reduction % for each scaffold, proceeding from the control (0 μg/ml) on the far left of each set of data through each increasing concentration and with the highest concentration (250 μg/ml) on the far right. Proliferation of keratinocytes on antimicrobial scaffolds followed the same trend as fibroblasts and decreased by the increase in concentration of silver in the coating solution.

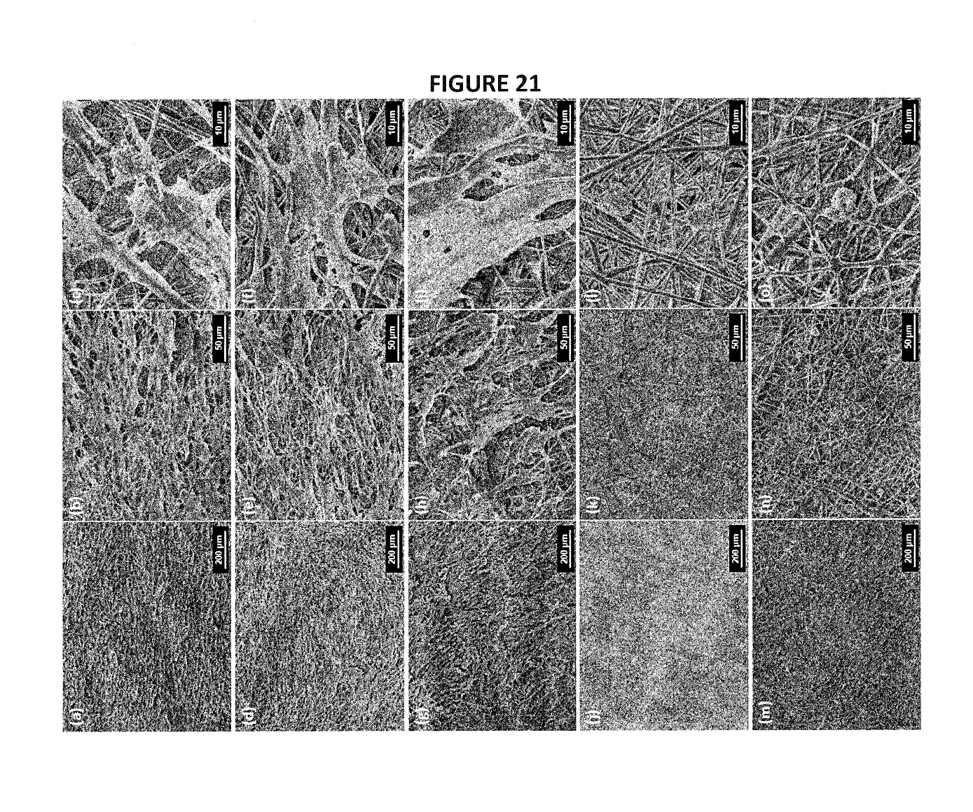

SEM micrographs indicated that fibroblast cells are adhered to the scaffolds. In FIG. 21, a, b, and c are SEM micrographs of fibroblast cells seeded on PLA bandages; d, e, and f are SEM micrographs of PLA bandages coated with Silvadur ET containing 31.25 μg/mL silver; g, h, and i are SEM micrographs of PLA bandages coated with Silvadur ET containing 62.5 μg/mL silver; j, k, and 1 are SEM micrographs of PLA bandages coated with Silvadur ET containing 125 μg/mL silver; and m, n, and o are SEM micrographs of PLA bandages coated with Silvadur ET containing 250 μg/mL silver. This figure shows that a large number of cells are spread throughout the surface of non-coated PLA nanofibers as well as the fibers coated with silvadur ET containing 31.25 and 62.5 μg/ml silver. However, for the nanofibers coated with silvadur ET containing 125 and 250 μg/ml silver, some cells on these scaffolds were rounded in shape, representative of the morphology of dead cells. These observations also confirm the results of viability analysis of cells. Antimicrobial efficacy is not diminished at a concentration of Silvadur ET that also maintains human skin cell viability and proliferation.

### Example 7

### Enhancement of Pore Formation/Control of Fiber Porosity

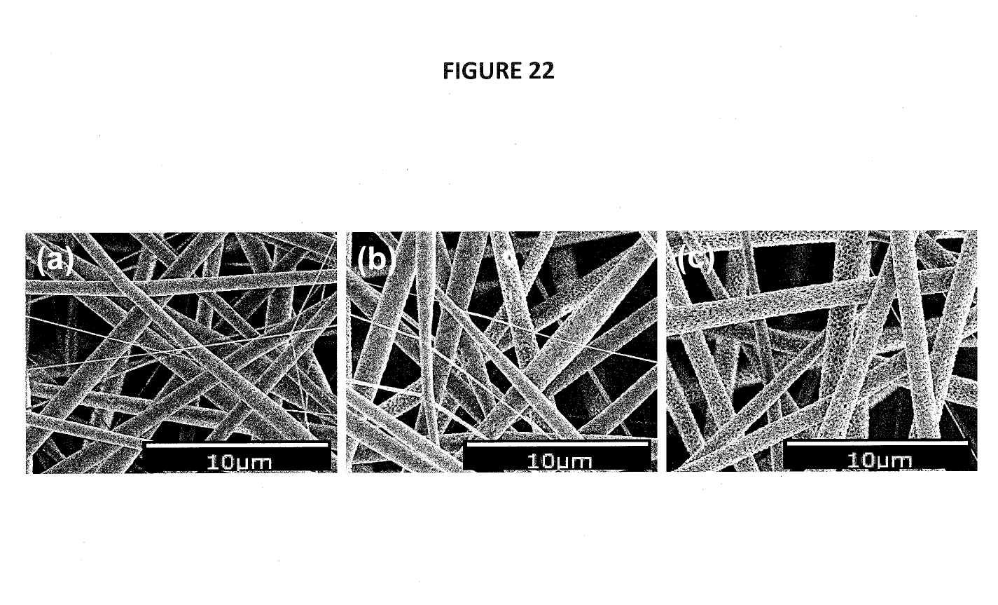

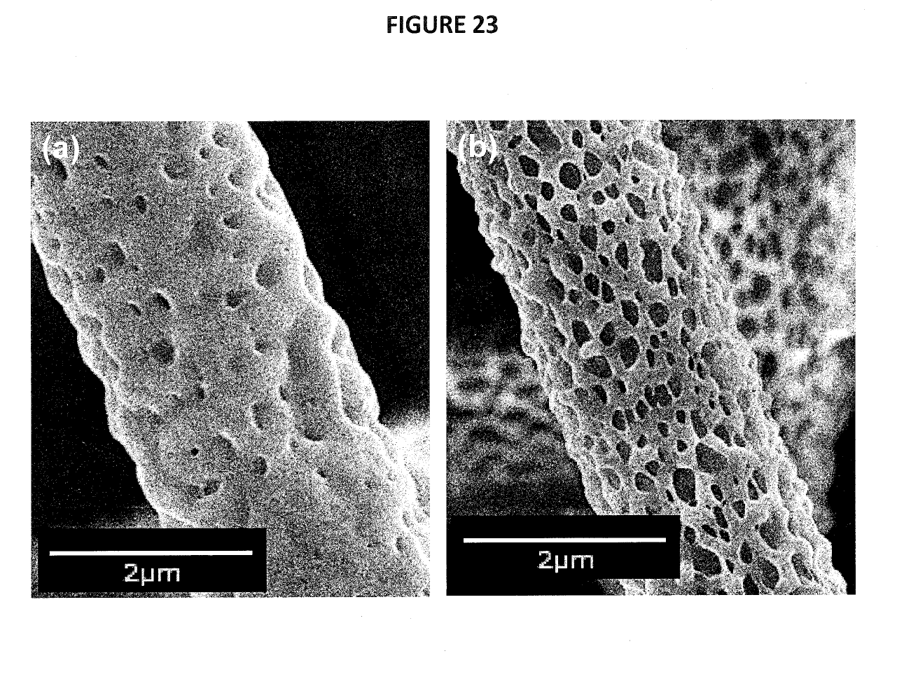

As described above, pore formation depends on the volatility of the solvent used for electro spinning and the humidity of the environment. Fibers were electrospun using dichloromethane as the solvent to spin porous fibers but changing the humidity of the spinning environment. Analyses of the surface morphology of fibers indicated that increasing humidity from 65% to 85% increased both the number (FIG. 22) and depth (FIG. 23) of pores formed. FIG. 22 provides SEM micrographs of porous fibers spun (a) in 65% relative humidity, (b) in 75% relative humidity, and (c) in 85% relative humidity. FIG. 23(a) is an SEM micrograph of a porous fiber spun in 75% relative humidity and FIG. 23(b) is an SEM micrograph of a porous fiber spun in 85% relative humidity.

### Example 8

### Development of Anti-Inflammatory Scaffolds for In Vivo Models

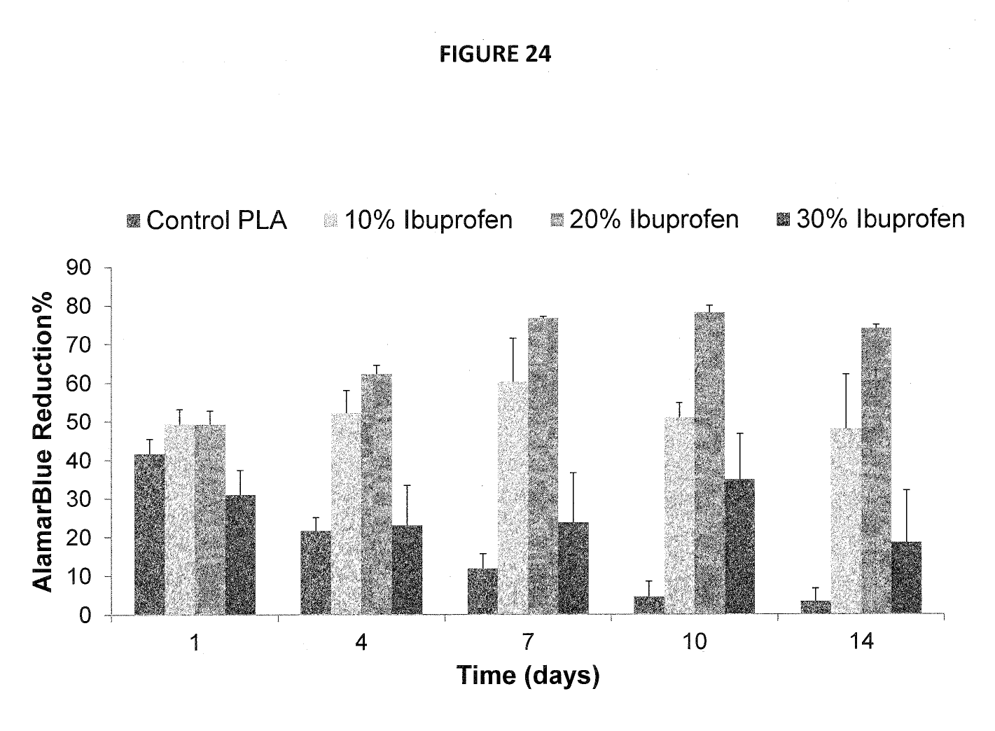

Poly(L-lactic acid) (PLA) was dissolved in dimethylformamide (DMF) and chloroform in a 1:3 ratio. (S)-(+)-Ibuprofen (Sigma-Aldrich, city state), was added to the PLA solution at concentrations of 10, 20 and 30 wt % ibuprofen relative to polymer weight. The solution was electrospun at 13-15 kV in room temperature and the resultant fibers punched into circles with an area of 2 cm². Surface morphology of the fibers was characterized using SEM and nuclear magnetic resonance (NMR) was used to confirm chemical composition of the fibers. Scaffolds were then treated with plasma to increase their hydrophilicity. Treated samples were placed in keratinocyte growth medium (KGM) for 12 hr and then seeded with human skin keratinocytes at a cell seeding density of 20K/cm². Proliferation of cells on both control PLA and ibuprofen loaded scaffolds was analyzed using an Alamar Blue assay and the results analyzed using a UV/Vis spectrophotometer to determine metabolic activity. Increasing ibuprofen up to 20% increases human keratinocyte proliferation relative to the PLA control with the highest proliferation occurring on PLA scaffolds doped with 20% ibuprofen. In FIG. 24, increases in AlamarBlue Reduction indicate greater cellular metabolic activity and are indicative of increased proliferation. FIG. 24 shows the AlamarBlue reduction % for each scaffold, proceeding from the control (control PLA) on the far left of each set of data through each increasing ibuprofen concentration and with the highest concentration (30%) on the far right.

### Example 9

### Comparison of Silver Nanoparticles and Silver Microparticles in PLA Nanofibers Scaffold Fabrication

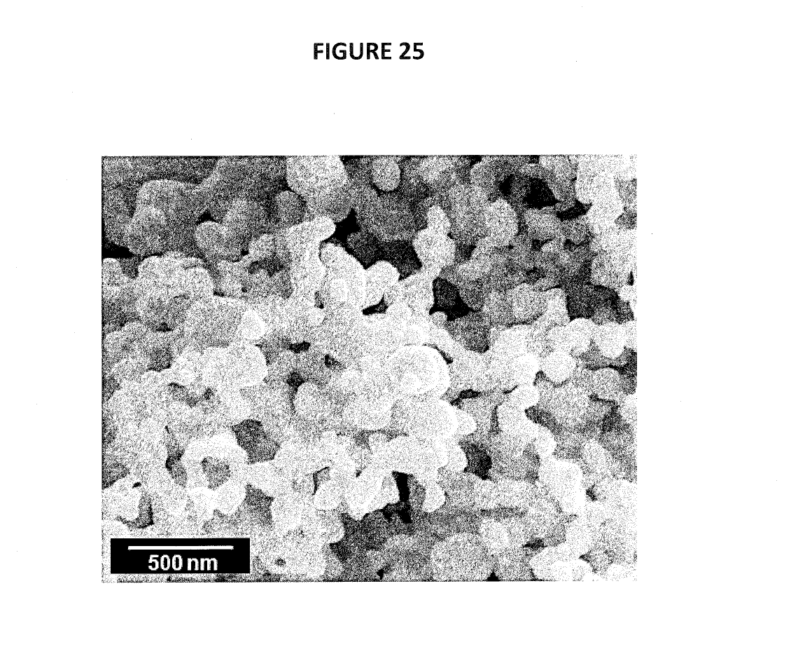

Polylactic acid (molecular weight=70000 g/mol) was dissolved in chloroform and dimethyl formamide (DMF, Sigma, St Louis, Mo., USA) at a ratio of 3 to 1 to create a 12% solution. Silver nanoparticles (20 nm Citrate Biopure™Silver) (Nanocomposix Company, San Diego, Calif., USA) or highly porous silver microparticles (average diameter=6.9 μm, surface area=3.9 m²/g) (BioGate Company, Nuremberg, Germany), shown in FIG. 25, were added to the PLA solution to obtain a 0.5% concentration silver to polymer ratio. Mixtures were stirred on a magnetic stirrer plate for 4 hours at 80° C. and then sonicated for 30 minutes to further ensure particle dissolution. Polymer solutions were used immediately after sonication to eliminate particle precipitation (of particular importance for microparticles) and prevent evaporative loss of solvent and consequent change in solution concentration. The PLA solution was electrospun for two hours using 15 kV voltage, feed rate of 0.7 μl/hr and spinning distance of 13-15 cm.

Electrospun scaffolds were kept under a fume hood overnight to fully evaporate residual solvents. Scaffolds were then removed from the fiber collector and cut into circles using a punch (d=1.6 cm). Scaffolds were sterilized with ethanol for 10 minutes and then rinsed three times with phosphate buffered saline (PBS) and soaked in keratinocyte growth media (KGM-Gold, Lonza, USA) without antibiotics for 12 hours to allow proteins to attach to the scaffolds. Pure PLA scaffolds containing no silver were used as controls.

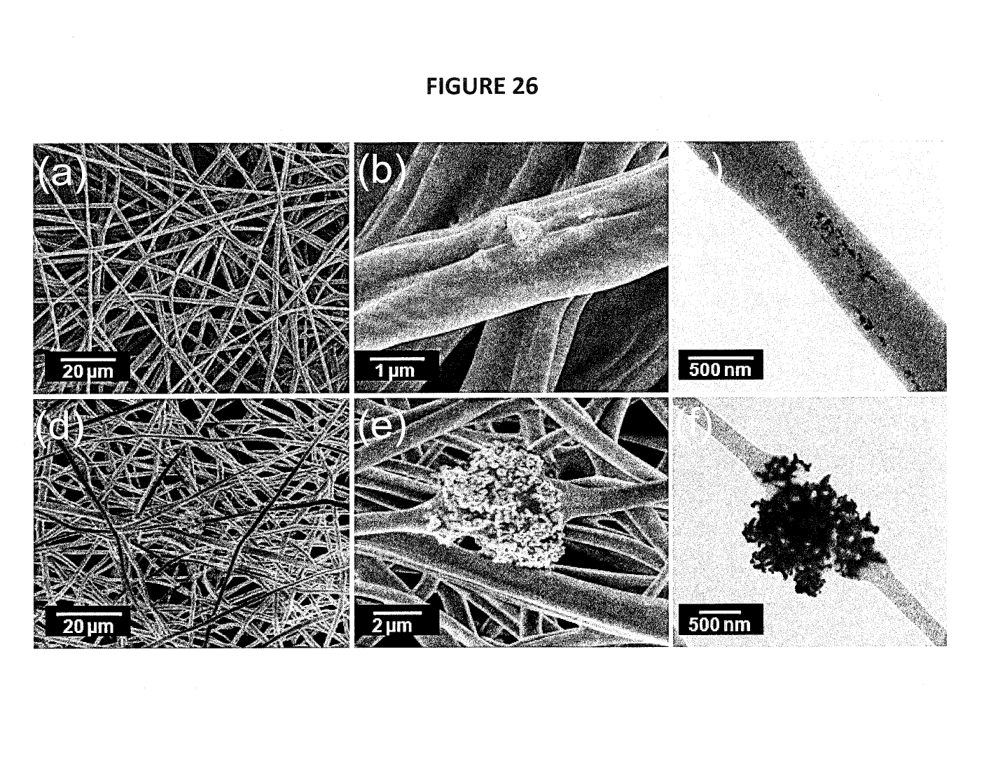

Electrospun PLA fibers containing either silver nanoparticles or highly porous microparticles formed in a uniform manner on the fiber collector (FIGS. 26(a) and (d)). However, as expected, incorporation of the two particles within the fibers varied. SEM images indicated that silver nanoparticles were not present on the surface of fibers and there were very few locations where nanoparticles were close to the fiber surface (FIG. 26(b)). TEM analysis confirmed the presence of well-dispersed nanoparticles inside the fibers, closer to the core (FIG. 26(c)). Highly porous silver microparticles, on the other hand, were too large to have been encapsulated inside the nanofibers. SEM (FIG. 26(e)) and TEM (FIG. 26(f)) images showed that these particles were present on the surface of fibers.

### Silver Release Studies

The electrospun scaffolds were soaked in deionized water and incubated at 37° C. and 5% CO₂ for one week. At specific time points: 3, 6, 18, 30, 42, 66, 120 and 168 hours, half of the water was removed and replaced with fresh deionized water. The concentration of silver ions released at each time point was quantified using the removed water via a Perkin-Elmer AA300 atomic adsorption spectrophotometer (AAS) (PerkinElmer Inc. Waltham, Mass.).

Co-Culture System of Human Epidermal Keratinocytes with S. aureus

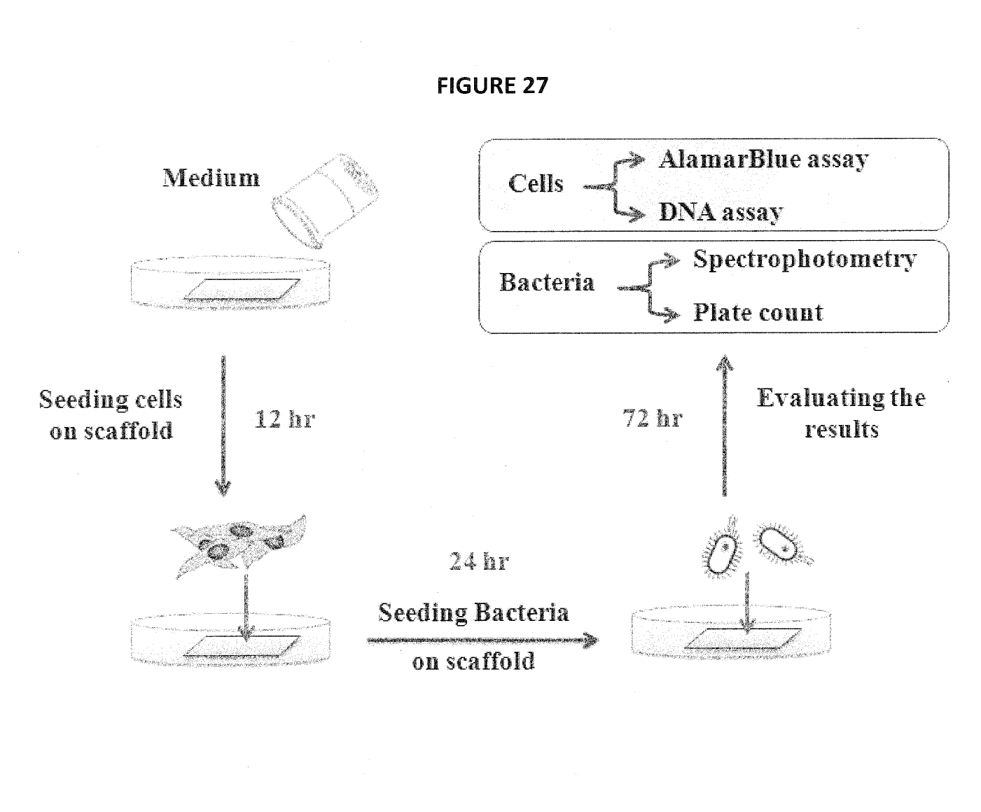

To evaluate the antimicrobial efficacy of silver nano- or microparticle loaded scaffolds when seeded with cells, a co-culture system and experimental process was designed, as illustrated in FIG. 27. Separate experiments with only human epidermal keratinocytes or S. aureus bacteria were also performed to better understand and elucidate the results of the co-culture system (Table 11, below). PLA scaffolds loaded with silver microparticles, silver nanoparticles, or neither (pure PLA, controls) were seeded with human epidermal keratinocytes derived from adult skin (2^{nd} passage, Lonza, USA) at a density of 4×10⁴ cells per scaffold and incubated for 24 hours. The cell seeded scaffolds were then inoculated with 10 CFU/ml S. aureus bacteria (AATCC#43300™) dispersed in keratinocyte growth media without antibiotics. Epidermal keratinocyte- and S. aureus-seeded scaffolds were then placed and maintained in an incubator (37° C., 5% CO₂) for 72 hours on a rotating plate, assisting with the suspension of bacteria.

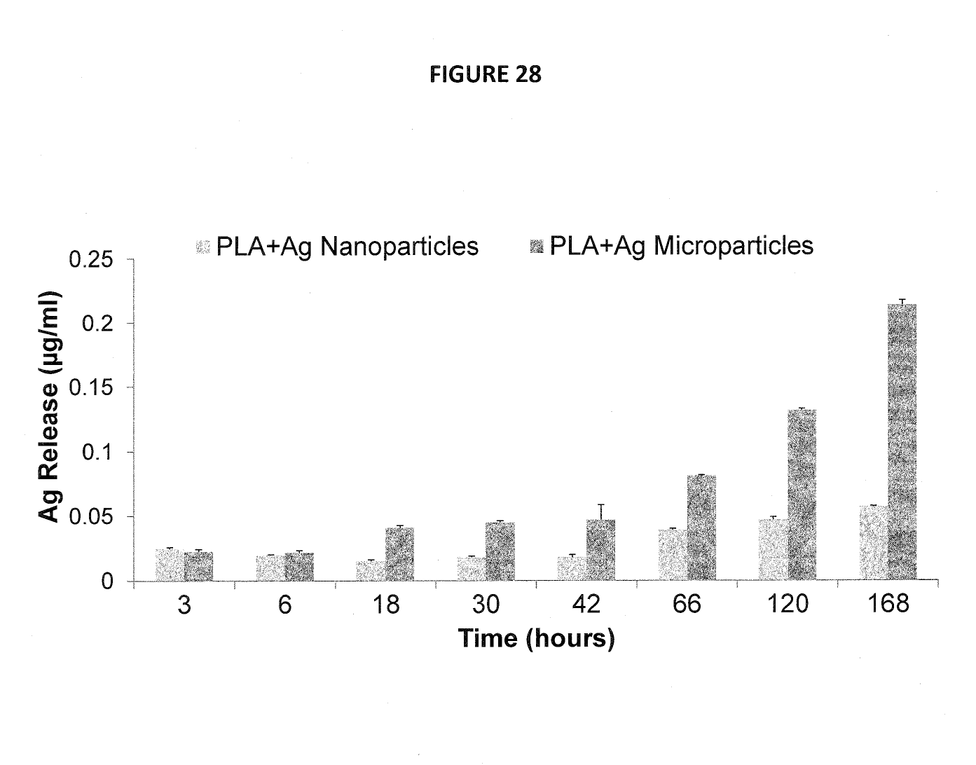

Release profiles revealed that silver ions gradually release from both scaffolds, doped with silver nanoparticles or highly porous silver microparticles, over a one week period (FIG. 28). Silver ion release was slow at the first two time points (3 and 6 hours) for both scaffolds, then significantly accelerated at the 16 hour time point for nanofibers doped with highly porous silver microparticles (FIG. 28). Rate of release and cumulative release remained greater for scaffolds doped with highly porous silver microparticles at all remaining time points.

TABLE 11
Design of experiment for antimicrobial and cytotoxicity evaluation
of scaffolds either separately or in a co-culture system

| | Scaffold | Replicates | Analyses |
|---|---|---|---|
| Cytotoxicity | Pure PLA | 3 | Alamar blue assay |
| experiment | PLA + Ag nanoparticles | | DNA assay |
| | PLA + Ag microparticles | | |
| Antimicrobial | Pure PLA | 3 | Spectrophotometry |
| experiment | PLA + Ag nanoparticles | | Plate count |
| | PLA + Ag microparticles | | |
| Co-culture | Pure PLA | 3 | Spectrophotometry |
| experiment | PLA + Ag nanoparticles | | Plate count |
| | PLA + Ag microparticles | | DNA assay |

Monitoring S. aureus Growth in Co-Culture System

At specific time points (12, 24, 26, 42, and 72 hours), 100 μl of culture medium was taken (after pipetting up and down several times to ensure bacteria were well suspended in the medium), diluted multiple times, and spread on Muller-Hinton (MH) agar plates (Thermo Scientific, USA). Agar plates were incubated overnight to allow bacterial colonies to grow and become visible to the naked eye for counting and monitoring bacterial growth in each well. After 72 hours, bacterial counts were confirmed by measurement of the optical density of the bacterial suspension, taken from each sample, using a UV-vis spectrophotometer (Biomate3, Thermo Electron Corporation, Madison, Wis., USA) at a wavelength of 600 nm.

### Human Epidermal Keratinocyte Viability and Proliferation in Co-Culture System

Viability analyses were performed at day 1 (24 hours after keratinocyte seeding on the scaffolds and prior to addition of bacteria) and day 3 (72 hours after addition of bacteria) using a fluorescent method (Live-Dead Assay Cytotoxicity Kit for mammalian cells; Molecular Probes, Eugene, Oreg.). Specifically, keratinocyte-seeded scaffolds were placed in KGM with the addition of 4 mM calceinacetoxymethyl ester-AM (staining the cytoplasm of live cells green) and 4 mM ethidiumhomodimer (staining the nuclei of dead cells red). The samples were then incubated for 20 minutes while protected from light. Cell proliferation, in cultures without bacteria, was determined with a cell viability assay (alamarBlue, AbDSerotec, Raleigh, N.C.) at different time points after seeding of cells on scaffolds (days 1, 2, and 3). AlamarBlue, at a volume of 10% of the culture medium, was added to each well 7 hours before each measurement. After incubation with alamarBlue, 200 μL of each sample was taken in triplicate and the absorbency read at 600 nm using a microplate reader (TecanGENios, Tecan, Switzerland). Greater alamarBlue reduction % indicated greater cell proliferation.

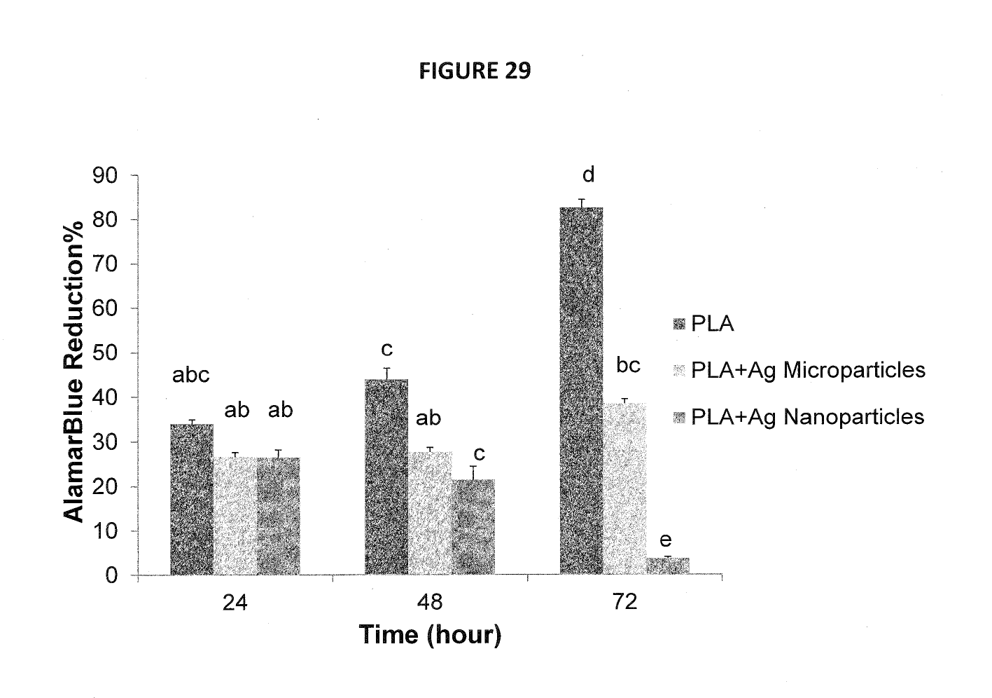

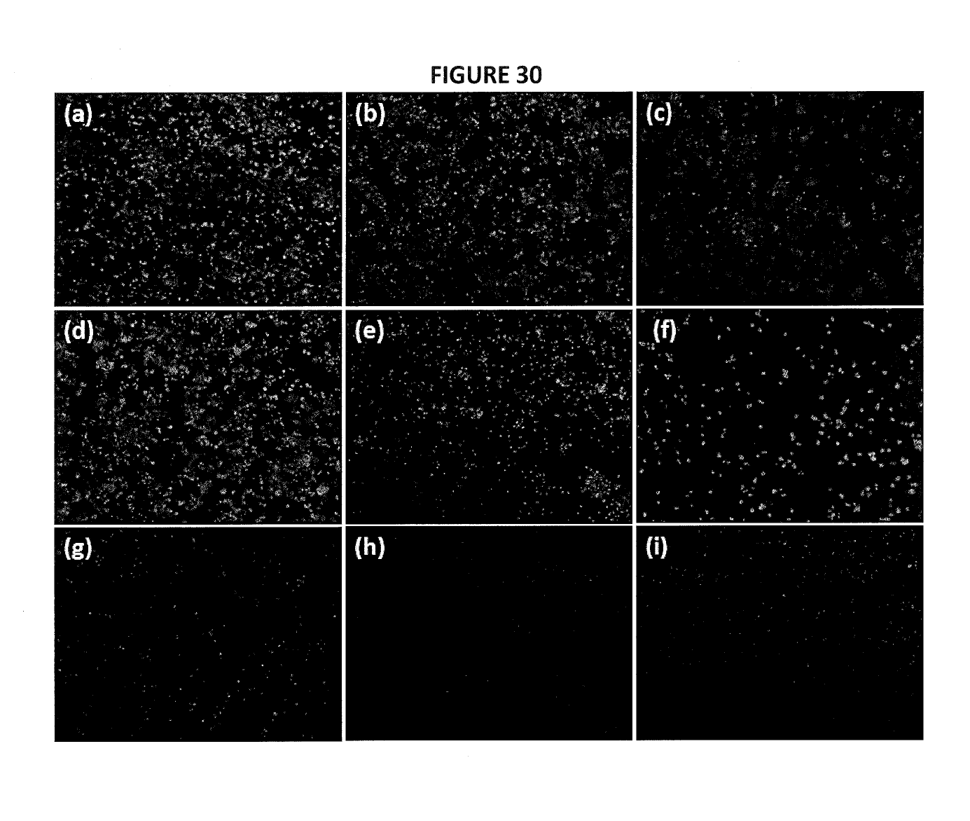

AlamarBlue findings indicated that proliferation of keratinocytes was diminished on PLA scaffolds loaded with silver nano- or microparticles compared to pure PLA scaffolds, with lowest proliferation observed for silver nanoparticle loaded scaffolds (FIG. 29). Similarly, viability analyses indicated that neither silver nano- or microparticle loaded scaffolds supported human epidermal keratinocyte viability after three days (FIG. 30). Addition of bacteria to the culture medium further decreased cell viability for all three keratinocyte-seeded scaffolds (FIG. 30).

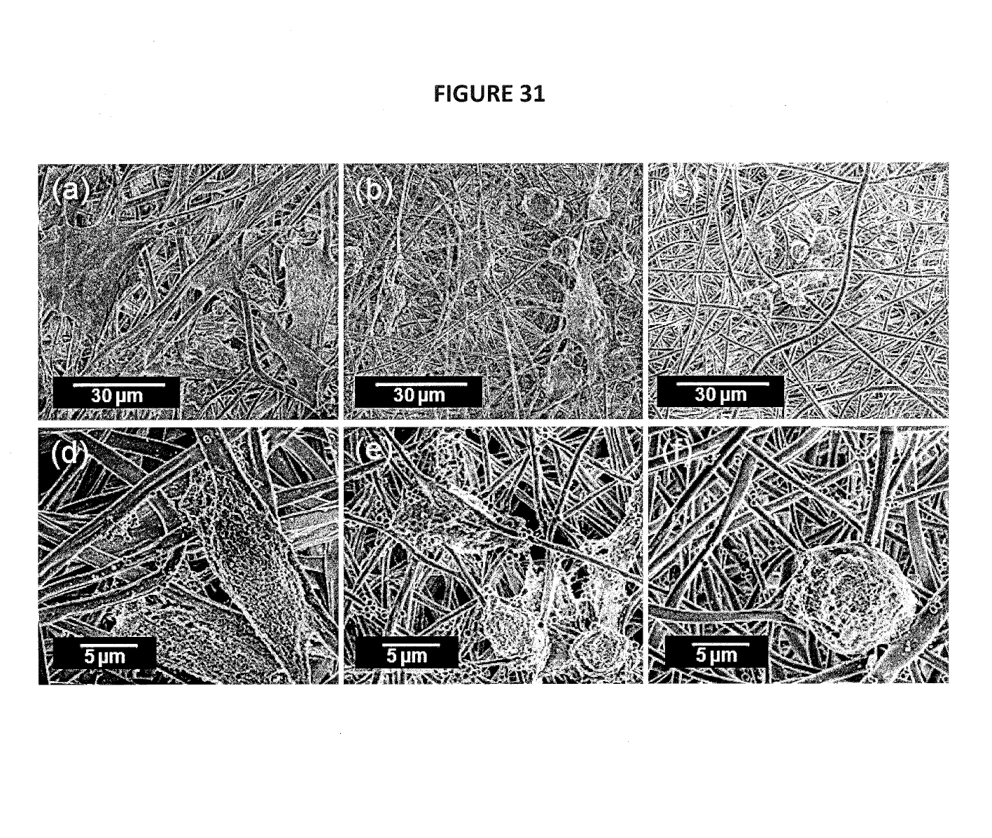

SEM micrographs showed that keratinocytes adhered and spread throughout the pure PLA scaffolds. However, keratinocyte morphology was clearly different on PLA scaffolds loaded with silver nano- or microparticles. On those scaffolds, cell number was reduced and cells were rounded, representing morphology of dead cells (FIG. 31). Consistent with qualitative viability analyses (FIG. 30), addition of bacteria to the culture medium further reduced the number of viable cells on pure PLA scaffolds. SEM images also confirmed presence of S. aureus bacteria with rounded morphology on the all three scaffolds.

Since AlamarBlue assay is not an appropriate method to evaluate the proliferation of cells in co-culture systems (presence of bacteria changes the reduction %), the number of cells on the scaffolds was quantified by measuring the DNA content in each scaffold after 72 hours. The scaffolds were washed at least three times with PBS to confirm that the bacteria were detached from the scaffolds. To verify that all bacteria were washed out from the scaffolds, the PBS solution from the last wash was used for bacterial analysis to confirm no bacteria were present in PBS from the last wash. Once this was confirmed, the amount of DNA in each nanofibrous scaffold was then measured with DNA binding dye Hoechst 33258 in microplate format after an overnight digestion at 60° C. in 2.5 units/mL papain in PBS with 5 mM ethylenediaminetetraacetic acid and 5 mM cysteine HCl (all reagents from Sigma).

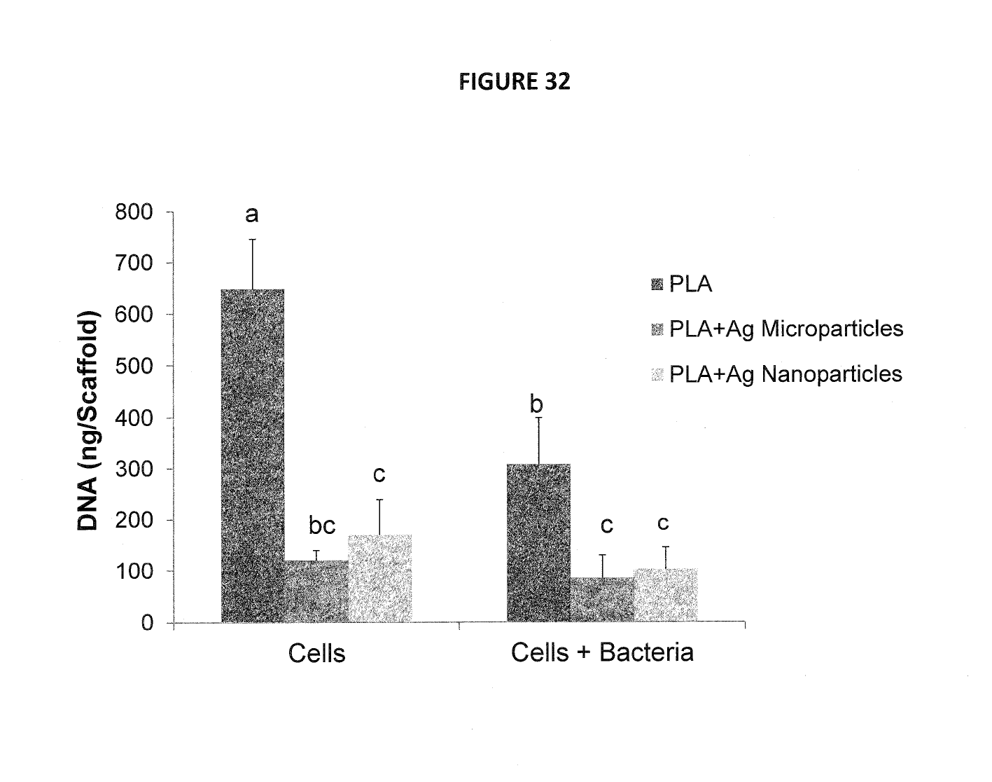

DNA quantitation (FIG. 32) indicated that pure PLA scaffolds supported the greatest keratinocyte viability relative to either silver nano- or microparticle loaded scaffolds in either the presence or absence of bacteria, consistent with viability (FIG. 30) and SEM (FIG. 31) analyses. DNA content significantly dropped for pure PLA scaffolds with the addition of bacteria to the culture medium.

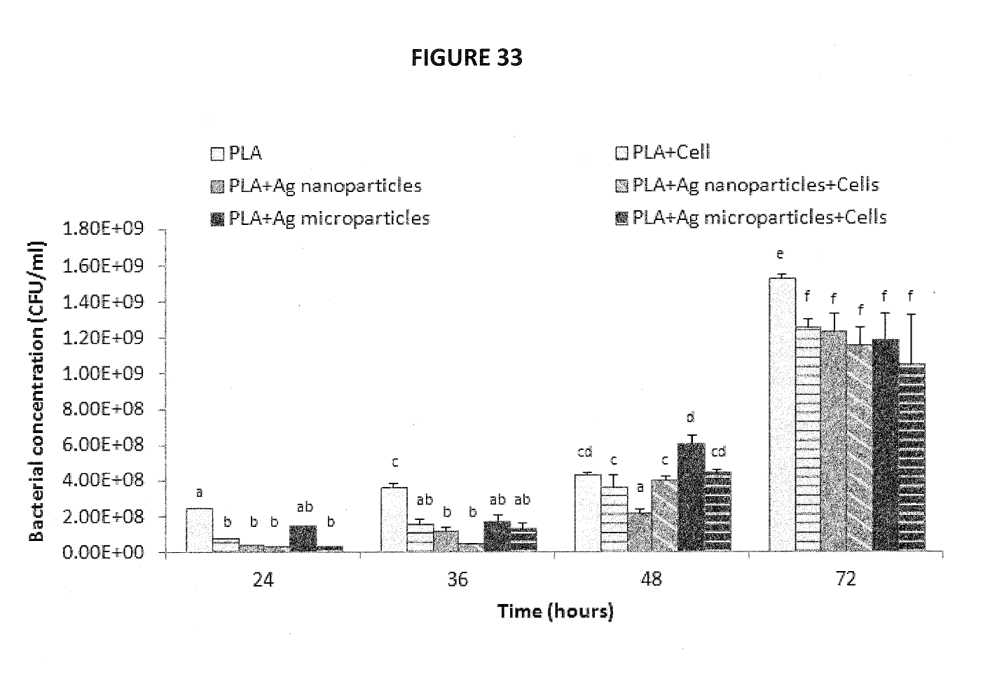

Bacterial analyses indicated that S. aureus proliferated on all three scaffolds, with the highest rate of growth for pure PLA scaffolds without keratinocytes (FIG. 33). These results not only indicate that addition of silver nano- or microparticles to the PLA scaffolds can reduce bacterial growth rate, but also that the presence of keratinocytes in co-culture systems with S. aureus diminishes bacterial growth.

### Microscopic Analyses

Keratinocyte morphology on the scaffolds was evaluated using scanning electron microscopy (SEM) (FESEM JEOL 6400 F) at 15 kV accelerating voltage. At each time point, nanofibrous scaffolds were fixed in 10% buffered formalin for 30 min and then dehydrated with a graded concentration (50-100% v/v) of ethanol. Dehydrated scaffolds were immersed in hexamethyldisilazane for 15 min and dried overnight in a fume hood. Dried samples were sputter coated with gold to observe the morphology of cells using SEM. Transmission electron microscopy (TEM) (Hitachi HF2000) was used to further characterize longitudinal dispersion of particles within ultra fine electrospun fibers at 200 kV accelerating voltage.

### Statistical Analyses

Statistical analyses were performed using SPSS 14.0. Data were analyzed using Duncan test with p-values less than 0.05 considered statistically significant.

Many modifications and other embodiments of the invention will come to mind to one skilled in the art to which this invention pertains having the benefit of the teachings presented in the foregoing description. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

1.-28. (canceled)

29. A multi-layered bandage comprising:
a first fibrous web comprising a plurality of electrospun hollow, porous fibers comprising one or more biocompatible polymers; and
a second fibrous web;
wherein at least one of the fibrous webs further comprises one or more therapeutic agents.

30. The multi-layered bandage of claim 29, wherein the one or more therapeutic agents are contained within the hollow portion of the electrospun fibers, coated on the surfaces of the electrospun fibers, or both contained within the hollow portion and coated on the surfaces of the electrospun fibers.

31. The multi-layered bandage of claim 29, wherein one therapeutic agent is contained within the hollow portion of the electrospun fibers and a second therapeutic agent is coated on the surfaces of the electrospun fibers.

32. The multi-layered bandage of claim 29, wherein the second fibrous web comprises a cellulosic material

33. The multi-layered bandage of claim 32, wherein the cellulosic material comprises cotton.

34. The multi-layered bandage of claim 29, wherein the one or more therapeutic agents are selected from the group consisting of antimicrobial agents, anti-inflammatories, growth factors, polysaccharides, proteins, collagen, oligonucleotides, cells, minerals, and combinations thereof.

35. The multi-layered bandage of claim 29, wherein the electrospun fibers comprise a biocompatible polymer selected from the group consisting of polylactic acid (PLA), polycaprolactone (PCL), polyethylene oxide (PEO), polyvinyl alcohol (PVA), polyglycolic acid (PGA), poly(ethylene-co-vinylacetate) (EVA), poly(ethyleneimine) (PEI), poly(2-hydroxyethyl methacrylate) (pHEMA), poly(2-hydroxypropyl methacrylate), poly(2-(dimethylamino)ethyl methacrylate), polylysine, poly(methylmethacrylate) (PMMA), polypyrroles, cyclodextrin, poly(a-[4-aminobutyl]-1-glycolic acid) (PAGA), poly(2-(dimethylamino)ethyl methacrylate) (pDMAEMA), poly(enol-ketone) (PEK), N-(2-hydroxypropyl)methacrylamide (HPMA), and blends, derivatives, and copolymers thereof.

36. The multi-layered bandage of claim 29, wherein the first fibrous web comprises two or more therapeutic agents.

37. A multi-layered bandage comprising:
a first fibrous web comprising a plurality of electrospun fibers comprising one or more biocompatible polymers and at least one of:
(i) one or more therapeutic agents imbedded in the fibers, coated on the surfaces of the fibers, or both imbedded in the fibers and coated on the surfaces of the fibers, wherein at least one therapeutic agent is a silver-containing therapeutic agent; and
(ii) one or more cellulosic materials imbedded in the fibers, coated on the surfaces of the fibers, or both imbedded in the fibers and coated on the surfaces of the fibers; and
a second fibrous web.

38. The multi-layered bandage of claim 37, wherein the electrospun fibers have a form selected from the group consisting of single-component fiber, core/sheath fiber, hollow fiber, porous fiber, and combinations thereof.

39. The multi-layered bandage of claim 37, wherein the silver-containing therapeutic agent comprises silver nanoparticles or silver microparticles.

40. The multi-layered bandage of claim 37, wherein the silver-containing therapeutic agent comprises silver microparticles having an average diameter of between about 1 micron and about 10 microns.

41. The multi-layered bandage of claim 37, wherein the silver-containing therapeutic agent comprises silver microparticles having an average surface area of at least about 2 m²/g.

42. The multi-layered bandage of claim 37, wherein the silver nanoparticles or silver microparticles are imbedded within the fibers.

43. The multi-layered bandage of claim 37, wherein the second fibrous web comprises a second cellulosic material.

44. The multi-layered bandage of claim 43, wherein the cellulosic material comprises cotton.

45. The multi-layered bandage of claim 37, wherein the cellulosic material is present in an amount of between about 5% and about 40% by weight of the fiber.

46. The multi-layered bandage of claim 37, wherein the cellulosic material is in the form of particles with a largest average dimension of less than about 500 microns.

47. The multi-layered bandage of claim 46, wherein the particles have a largest average dimension of between about 200 and about 400 microns.

48. The multi-layered bandage of claim 37, comprising both (i) one or more therapeutic agents and (ii) one or more first cellulosic materials.

49. The multi-layered bandage of claim 37, wherein the bandage comprises two or more therapeutic agents.

50. The multi-layered bandage of claim 37, wherein the first fibrous web comprises two or more therapeutic agents.

51. The multi-layered bandage of claim 37, wherein the electrospun fibers comprise a biocompatible polymer selected from the group consisting of polylactic acid (PLA), polycaprolactone (PCL), polyethylene oxide (PEO), polyvinyl alcohol (PVA), polyglycolic acid (PGA), poly(ethylene-co-vinylacetate) (EVA), poly(ethyleneimine) (PEI), poly(2-hydroxyethyl methacrylate) (pHEMA), poly(2-hydroxypropyl methacrylate), poly(2-(dimethylamino)ethyl methacrylate), polylysine, poly(methylmethacrylate) (PMMA), polypyrroles, cyclodextrin, poly(a-[4-aminobutyl]-1-glycolic acid) (PAGA), poly(2-(dimethylamino)ethyl methacrylate) (pDMAEMA), poly(enol-ketone) (PEK), N-(2-hydroxypropyl)methacrylamide (HPMA), and blends, derivatives, and copolymers thereof.

52. A fibrous web comprising a plurality of electrospun hollow, porous fibers comprising one or more biocompatible polymers, wherein the fibers further comprise two or more therapeutic agents exhibiting different rates of release from the fibrous web.

53. The fibrous web of claim 52, wherein the two or more therapeutic agents comprise a first therapeutic agent located within the hollow portion of the fibers and a second therapeutic agent located within pores on the surface of the fibers, incorporated in the one or more biocompatible polymers, or coated onto the exterior of the fibers, and wherein the first therapeutic agent exhibits slower release from the fibrous web than the second therapeutic agent.

54. The fibrous web of claim 53, wherein the first therapeutic agent comprises an antimicrobial agent and the second therapeutic agent comprises an agent selected from the group consisting of antimicrobial agents, anti-inflammatories, growth factors, polysaccharides, proteins, collagen, oligonucleotides, cells, minerals, and combinations thereof.
